(19) United States
(12) Patent Application Publication
(10) Pub. No.: US 2015/0374760 A1
(43) Pub. Date: Dec. 31, 2015

(54) METHODS FOR TREATING PSORIASIS AND PSORIATIC ARTHRITIS

(71) Applicants: Jose U. Scher, Jersey City, NJ (US); Carles Ubeda, Valencia (ES); Dan R. Littman, New York, NY (US); Eric G. Pamer, Guilford, CT (US); Steven B. Abramson, Rye, NY (US)

(72) Inventors: Jose U. Scher, Jersey City, NJ (US); Carles Ubeda, Valencia (ES); Dan R. Littman, New York, NY (US); Eric G. Pamer, Guilford, CT (US); Steven B. Abramson, Rye, NY (US)

(21) Appl. No.: 14/682,344

(22) Filed: Apr. 9, 2015

Related U.S. Application Data
(60) Provisional application No. 62/064,664, filed on Oct. 16, 2014.
(60) Provisional application No. 61/977,272, filed on Apr. 9, 2014.

(51) Int. Cl. A61K 35/74

(52) U.S. Cl. CPC A61K 35/74

(57) ABSTRACT

Methods, agents and compositions thereof for treating psoriatic arthritis (PsA) and psoriasis (Ps) are encompassed herein, as are methods for identifying pathogenic dysbiosis in a subject, the presence of which is a positive indicator of risk for developing PsA or of the presence of PsA in a subject with arthritic symptoms.

### CROSS REFERENCE TO RELATED APPLICATIONS

This application claims priority under 35 USC §119(e) from U.S Provisional Application Ser. No. 61/977,272, filed Apr. 9, 2014, and U.S Provisional Application Ser. No. 62/064,664, filed Oct. 16, 2014, each of which applications is herein specifically incorporated by reference in its entirety.

### GOVERNMENTAL SUPPORT

The research leading to the present invention was supported, at least in part, by RC2 AR058986 and K23AR064318 from the National Institute of Arthritis and Musculoskeletal and Skin Diseases. Accordingly, the Government has certain rights in the invention.

### FIELD OF THE INVENTION

Therapeutic, diagnostic, and prognostic methods pertaining to psoriasis (Ps) and psoriatic arthritis (PsA) are described herein. Compositions for use in such methods are also set forth herein.

### BACKGROUND OF THE INVENTION

Spondylarthritides (SpA) are disabling rheumatic diseases that present mainly with inflammation of the axial skeleton, peripheral joints, and tendons. PsA is a type of chronic SpA, typically affecting individuals with pre-existing psoriasis of the skin (Ps). Psoriasis is a chronic, genetically based, immune-mediated inflammatory disorder affecting 2%-3% of the Caucasian population in western countries (Nestle et al. N Engl J Med 361:496-509, 2009). Psoriasis plaques may be localized or widespread across the body, and fingernails and toenails are frequently involved (Puig et al. Clinicoecon Outcomes Res. 6: 93-100, 2014).

PsA has been defined as a unique inflammatory arthritis associated with psoriasis. It is viewed as a complex disease in which environmental, host, and random factors coalesce, leading to disease in genetically susceptible individuals (Gladman et al. Ann Rheum Dis. 64(Suppl 2): ii14-ii17, 2005; Queiro et al. ISRN Dermatol. 2014; 2014: 570178.). PsA is associated with significant morbidity and mortality and is estimated to incur costs equivalent to those of rheumatoid arthritis (Zink et al. Journal of Rheumatology 33:86-90, 2006). Despite recent advances in diagnosis and treatment, however, the pathogenesis of PsA remains unclear. Previously proposed environmental factors that may trigger disposition to or development of PsA include viruses, vaccinations, bacterial infections, trauma and stress.

In view of the above, new methods for use in the accurate diagnosis, prognosis, and/or monitoring of patients with PsA are urgently needed. Methods described herein address these needs.

The citation of references herein shall not be construed as an admission that such is prior art to the present invention.

### SUMMARY OF THE INVENTION

The present inventors have characterized the abundance and diversity of gut microbiota in patients with never-treated, new-onset psoriatic arthritis (PsA). To achieve this end, high-throughput 16S rRNA pyrosequencing was utilized to compare community composition of gut microbiota in PsA patients (n=16), subjects with psoriasis of the skin (Ps) (n=15) and healthy, matched-controls (n=17). Samples were further assessed for the presence and levels of fecal and serum secretory immunoglobulin A (sIgA), and pro-inflammatory proteins. Samples from patients with PsA, psoriasis of the skin (Ps), new-onset rheumatoid arthritis (NORA), and healthy controls were also assessed for the presence and levels of fecal secretory immunoglobulin A (sIgA) and various proteins and pro-inflammatory cytokines.

As described in detail herein, gut microbiota or gut microbiome profile observed in PsA and Ps patients was less diverse when compared to healthy controls. These microbial differences were attributed to the reduced presence of several taxa in the PsA intestinal microbiota. While both groups showed a relative decrease in Coprococcus species, PsA samples were characterized by a significant reduction in, for example, the genera of Akkermansia, Ruminococcus, and Pseudobutyrivibrio. Supernatants of fecal samples from PsA patients also revealed an increase in sIgA and a decrease in receptor activator of nuclear factor kappa-B ligand (RANKL) levels.

In an aspect of the present discoveries, a method for treating a subject afflicted with psoriasis is presented, the method comprising administering to the subject a therapeutically effective amount of a sample comprising a fecal transplant isolated from a healthy donor or a therapeutically effective amount of a composition comprising at least one purified bacterial species of a genera of Akkermansia, Ruminococcus, Pseudobutyrivibrio, Coprococcus, and Coprobacillus, wherein administering the fecal transplant or the composition comprising the at least one purified bacterial species treats the subject afflicted with psoriasis. In a particular embodiment thereof, the composition comprises a sterile physiologically compatible carrier or excipient.

In an embodiment thereof, the subject afflicted with psoriasis has decreased bacterial diversity of gut microbiota. In a more particular embodiment, the subject afflicted with psoriasis exhibits patches of thick, inflamed skin covered with silvery scales. These patches, or plaques, are usually itchy. They most often occur on the elbows, knees, other parts of the legs, scalp, lower back, face, palms, and soles of the feet, but they can occur on skin anywhere on the body. The disease may also affect the fingernails, the toenails, and the soft tissues of the genitals, and inside the mouth. A subject afflicted with psoriasis does not exhibit abdominal pain, vomiting, diarrhea, rectal bleeding, severe pelvic cramps and/or weight loss, which symptoms are characteristic of inflammatory bowel disease (IBD); and does not exhibit pain, swelling, or stiffness in one or more joints, joints that are red or warm to the touch, sausage-like swelling in the fingers or toes (i.e., dactylitis), pain in and around the feet and ankles, especially tendinitis in the Achilles tendon or Plantar fasciitis in the sole of the foot, and/or lower back pain (i.e., in the area of the sacrum or sacroiliitis), symptoms characteristic of PsA.

In another embodiment, the subject afflicted with psoriasis has psoriatic arthritis. In a more particular embodiment, the subject afflicted with psoriatic arthritis exhibits psoriasis of the skin associated with: pain, swelling, or stiffness in one or more joints, joints that are red or warm to the touch, sausage-like swelling in the fingers or toes (i.e., dactylitis), pain in and around the feet and ankles, especially tendinitis in the Achilles tendon or Plantar fasciitis in the sole of the foot, or lower back pain (i.e. in the area of the sacrum or sacroiliitis); and does not exhibit abdominal pain, vomiting, diarrhea, rectal bleeding, severe pelvic cramps and/or weight loss (symptoms characteristic of IBD).

In yet another embodiment, the healthy donor is an individual without history of any chronic medical condition. In a particular embodiment thereof, healthy donors are free of known enteropathogens.

In a further embodiment, the sample comprising the fecal transplant is administered orally or anally. In a more particular embodiment, the fecal transplant is administered anally into at least one of the terminal ileum and right colon.

In another embodiment, the at least one pure or purified bacterial species is selected from the group consisting of Akkermansia muciniphila (A. muciniphila), Ruminococcus albus (R. albus), Ruminococcus callidus (R. callidus), Ruminococcus bromii (R. bromii), and Ruminococcus gnavus (R. gnavus). In a more particular embodiment, the at least one purified bacterial species is R. gnavus. In a more particular embodiment, the at least one purified bacterial species is the Coprococcus species designated ATCC®27761™; ATCC®27758™; or ATCC®27759™; the Ruminococcus species Ruminococcus gnavus (ATCC®BAA-35913™) or Ruminococcus gnavus (ATCC®BAA-29149™); and/or the Akkermansia species Akkermansia muciniphila (ATCC®BAA-835™). In an even more particular embodiment, the at least one purified bacterial species is the Akkermansia species Akkermansia muciniphila (ATCC®BAA-835); the Coprococcus species designated ATCC®29549™; and/or the Ruminococcus species Ruminococcus gnavus (ATCC®BAA-35913™) or Ruminococcus gnavus (ATCC®BAA-29149™). In another particular embodiment, the at least one purified bacterial species is Ruminococcus gnavus (ATCC®BAA-35913™) or Ruminococcus gnavus (ATCC®BAA-29149™). In a further particular embodiment, wherein the at least one purified bacterial species comprises a combination of purified A. muciniphila and purified R. gnavus; a combination of purified A. muciniphila, purified R. gnavus, and a purified species of Coprococcus; a combination of purified A. muciniphila, purified R. gnavus, and a purified species of Pseudobutyrivibrio, or a combination of purified A. muciniphila, purified R. gnavus, a purified species of Coprococcus, and a purified species of Pseudobutyrivibrio.

Also encompassed herein is a method for identifying pathogenic microbial dysbiosis in a subject, the method comprising providing or selecting a subject afflicted with psoriasis (Ps) or a subject afflicted with arthritic symptoms; isolating a fecal sample from the subject and processing the fecal sample to generate a fecal bacterial sample; analyzing microbiota diversity in the fecal bacterial sample, wherein a decrease in at least one of a bacterial species of Pseudobutyrivibrio, Akkermansia, Ruminococcus, Coprococcus, Coprobacillus, Unclassified (UC)_{—}Clostridia, Verrucomicrobiae, Verrucomicrobia, and Verrucomicrobiales identifies the subject afflicted with psoriasis or the subject afflicted with arthritic symptoms as having pathogenic microbial dysbiosis, wherein the presence of pathogenic microbial dysbiosis is a positive indicator of risk for developing psoriatic arthritis (PsA) in the subject afflicted with psoriasis or of having PsA in the subject afflicted with arthritic symptoms. In a particular embodiment thereof, the decrease in the at least one bacterial species is determined relative to that of a healthy subject or donor. In another embodiment, the subject afflicted with Ps has arthritic symptoms. In yet another embodiment, the method further comprises assessing receptor activator of nuclear factor kappa-B ligand (RANKL) levels in the subject, wherein identification of pathogenic microbial dysbiosis in combination with reduced RANKL levels strengthens the determination that the subject with Ps is at risk for developing PsA or the subject with arthritic symptoms has PsA and should, therefore, be treated accordingly.

In an embodiment thereof, the method further comprises treating the subject identified as having pathogenic microbial dysbiosis by administering to the subject a therapeutically effective amount of a sample comprising a fecal transplant isolated from a healthy donor or a therapeutically effective amount of a composition comprising at least one purified bacterial species of Akkermansia, Ruminococcus, Pseudobutyrivibrio, Coprococcus, Coprobacillus, Unclassified (UC)_{—}Clostridia, Verrucomicrobiae, Verrucomicrobia, and Verrucomicrobiales, wherein administering the fecal transplant or the composition treats the subject by reducing the degree of microbial dysbiosis in the subject. In a particular embodiment thereof, the composition comprises a sterile physiologically compatible carrier or excipient.

In a particular embodiment thereof, the composition comprises at least one purified bacterial species selected from the group consisting of Akkermansia muciniphila (A. muciniphila), Ruminococcus albus (R. albus), Ruminococcus callidus (R. callidus), Ruminococcus bromii (R. bromii), Ruminococcus gnavus (R. gnavus), and Coprobacillus cateniformis (C. cateniformis).

In a more particular embodiment, the at least one purified bacterial species included in the composition is the Coprococcus species ATCC®27761™; ATCC®27758™; or ATCC®27759™; the Ruminococcus species Ruminococcus gnavus (ATCC®BAA-35913™) or Ruminococcus gnavus (ATCC®BAA-29149™); the Coprobacillus species Coprobacillus cateniformis (DSMZ No. 15921), and/or the Akkermansia species Akkermansia muciniphila (ATCC®BAA-835™). In an even more particular embodiment, the at least one purified bacterial species included in the composition is the Coprococcus species designated ATCC®27761™; ATCC®27758™; or ATCC®27759™; and/or the Ruminococcus species Ruminococcus gnavus (ATCC®BAA-35913™) or Ruminococcus gnavus (ATCC®BAA-29149™). In another particular embodiment, the at least one purified bacterial species included in the composition is Ruminococcus gnavus (ATCC®BAA-35913 ™) or Ruminococcus gnavus (ATCC®BAA-29149™). In another particular embodiment, the composition comprises a combination of purified A. muciniphila and purified R. gnavus; a combination of purified A. muciniphila, purified R. gnavus, and a purified species of Coprococcus; a combination of purified A. muciniphila, purified R. gnavus, and a purified species of Pseudobutyrivibrio; or a combination of purified A. muciniphila, purified R. gnavus, a purified species of Coprococcus, and a purified species of Pseudobutyrivibrio.

In an embodiment thereof, the analyzing comprises nucleic acid sequencing. In a more particular embodiment, the nucleic acid sequencing is shotgun sequencing. Targeted sequencing methods are also encompassed herein. Such methods would focus sequencing analyses to 16S rRNAs specific for the bacterial taxa and species identified as underrepresented in Ps and/or PsA patients at the OTU-species level. Detection of the aforementioned 16S rRNAs may also be achieved using probes specific for these sequences. Antibodies specific for the bacterial taxa and species described herein are also envisioned to assay and evaluate the microbiome profile of a subject.

Also encompassed herein is a composition for treating PsA in a subject, the composition comprising a therapeutically effective amount of at least one purified bacterial species of Akkermansia, Ruminococcus, Pseudobutyrivibrio, Coprococcus, Coprobacillus, Unclassified (UC)_{—}Clostridia, Verrucomicrobiae, Verrucomicrobia, and Verrucomicrobiales and a sterile manmade physiologically compatible carrier or excipient.

In a particular embodiment thereof, the sterile manmade physiologically compatible carrier or excipient is compatible with oral and/or anal administration. Exemplary buffers compatible with oral administration include sterile manmade solutions that are physiologically compatible such as, for example, sterile normal saline or a sterile saline-based gelatin or matrix. Normal saline is typically defined as a solution of 0.90% weight/volume of NaCl, about 300 mOsm/L or about 9.0 grams NaCl per liter of water. In a particular embodiment, the exemplary buffer comprises butyrate, fermentable fiber, and/or resistant starch and, more particularly, at least one of a Type 1-4 resistant starch. In a particular embodiment, oral administration is achieved using an encapsulated means, wherein the capsule is designed to dissolve or disintegrate in the small and/or large intestine. Exemplary buffers compatible with anal administration comprise sterile manmade solutions that are physiologically compatible such as, for example, normal saline, saline-based gelatin, oleaginous (fatty) bases [e.g., theobroma oil (cocoa butter) and synthetic triglycerides], and water soluble or miscible bases (e.g., glycerinated gelatin and polyethylene glycol polymers).

In a further embodiment thereof, the at least one purified bacterial species is selected from the group consisting of Akkermansia muciniphila (A. muciniphila), Ruminococcus albus (R. albus), Ruminococcus callidus (R. callidus), Ruminococcus bromii (R. bromii), and Ruminococcus gnavus (R. gnavus). In yet another embodiment, the composition comprises at least two purified bacterial species of a genera of Akkermansia, Ruminococcus, Pseudobutyrivibrio, Coprococcus, and Coprobacillus. In a further embodiment, the composition comprises at least three, four, five, six. or more purified bacterial species of a genera of Akkermansia, Ruminococcus, Pseudobutyrivibrio, Coprococcus, and Coprobacillus. More particularly, the Coprococcus species is ATCC®27761™; ATCC®27758™; or ATCC®27759™; the Ruminococcus species is Ruminococcus gnavus (ATCC®BAA-35913™) or Ruminococcus gnavus (ATCC®BAA-29149™); and/or the Akkermansia species is Akkermansia muciniphila (ATCC®BAA-835™). In yet another embodiment, the composition comprises a combination of purified A. muciniphila and purified R. gnavus; a combination of purified A. muciniphila, purified R. gnavus, and a purified species of Coprococcus; a combination of purified A. muciniphila, purified R. gnavus, and a purified species of Pseudobutyrivibrio; or a combination of purified A. muciniphila, purified R. gnavus, a purified species of Coprococcus, and a purified species of Pseudobutyrivibrio.

Also encompassed herein is a method for treating a subject afflicted with psoriatic arthritis, the method comprising administering to the subject a therapeutically effective amount of a sample comprising a fecal transplant isolated from a healthy donor or a therapeutically effective amount of a composition comprising at least one purified bacterial species of Akkermansia, Ruminococcus, Pseudobutyrivibrio, Coprococcus, Coprobacillus, Unclassified (UC)_{—}Clostridia, Verrucomicrobiae, Verrucomicrobia, and Verrucomicrobiales, wherein administering the fecal transplant or the composition treats the subject afflicted with psoriatic arthritis. In a particular embodiment thereof, the composition comprises a sterile physiologically compatible carrier or excipient.

Also encompassed herein is a method for treating a subject afflicted with psoriasis, the method comprising administering to the subject a therapeutically effective amount of a sample comprising a fecal transplant isolated from a healthy donor or a therapeutically effective amount of a composition comprising at least one purified bacterial species of Coprococcus, Coprobacillus, Porphyromonadaceae, Parabacteroides, Unclassified (UC)_{—}Clostridia, Erysipelotrichaceae, Erysipelotrichales, Erysipelotrichi, and Actinobacteria phylum, wherein administering the fecal transplant or the composition treats the subject afflicted with psoriasis. In a particular embodiment thereof, the composition comprises a sterile physiologically compatible carrier or excipient.

In a further embodiment thereof, the subject afflicted with psoriatic arthritis or psoriasis has decreased bacterial diversity of gut microbiota. As described herein, the subject afflicted with psoriatic arthritis typically exhibits psoriasis of the skin associated with: pain, swelling, or stiffness in one or more joints, joints that are red or warm to the touch, dactylitis, pain in and around the feet and ankles, and/or lower back pain and does not exhibit abdominal pain, vomiting, diarrhea, rectal bleeding, severe pelvic cramps and/or weight loss. A subject afflicted with psoriasis exhibits patches of thick, inflamed skin covered with silvery scales; and does not exhibit abdominal pain, vomiting, diarrhea, rectal bleeding, severe pelvic cramps and/or weight loss; and does not exhibit pain, swelling, or stiffness in one or more joints, joints that are red or warm to the touch, dactylitis, pain in and around the feet and ankles, and/or lower back pain.

In accordance with methods for treating a subject afflicted with PsA or Ps as described herein, a healthy donor is an individual without any chronic medical conditions and may also have been evaluated to determine that he/she is free of known enteropathogens; and the sample comprising the fecal transplant is administered orally or anally (e.g., into at least one of the terminal ileum and right colon). In an aspect thereof, the Coprococcus species is ATCC®27761™; ATCC®27758™; or ATCC®27759™.

Also encompassed herein is a composition for treating Ps in a subject, the composition comprising a therapeutically effective amount of at least one purified bacterial species of Coprococcus, Coprobacillus, Porphyromonadaceae, Parabacteroides, Unclassified (UC)_{—}Clostridia, Erysipelotrichi, and Actinobacteria phylum and a sterile physiologically compatible carrier or excipient. In an aspect thereof, the Coprococcus species is ATCC®27761™; ATCC®27758™; or ATCC®27759™. The composition may be compatible with oral and/or anal administration and may further comprise at least one of a resistant starch, fermentable fiber, and/or butyrate. In a particular embodiment, the composition comprises at least two purified bacterial species of Coprococcus, Coprobacillus Porphyromonadaceae, Parabacteroides, Unclassified (UC)_{—}Clostridia, Erysipelotrichi, and Actinobacteria phylum. In another particular embodiment, the composition comprises at least three, four, five, six, or more purified bacterial species of Pseudobutyrivibrio, Coprococcus, Coprobacillus Porphyromonadaceae, Parabacteroides, Unclassified (UC)_{—}Clostridia, Erysipelotrichi, and Actinobacteria phylum.

Also encompassed herein is a method for treating a subject afflicted with psoriasis (Ps) or psoriatic arthritis (PsA), the method comprising administering to the subject a therapeutically effective amount of receptor activator of nuclear factor kappa-B ligand (RANKL) or a composition thereof, wherein administering the therapeutically effective amount of RANKL or the composition thereof treats the subject afflicted with Ps or PsA. In an embodiment thereof, the RANKL or the composition thereof are administered orally or anally (e.g., into at least one of the terminal ileum and right colon). In a particular embodiment thereof, the composition comprises a sterile physiologically compatible carrier or excipient.

Other objects and advantages will become apparent to those skilled in the art from a review of the following description which proceeds with reference to the following illustrative drawings.

### BRIEF DESCRIPTION OF THE DRAWINGS

FIG. 1. Bacterial taxa differences between PsA, Ps and healthy controls. Bacterial taxa significantly enriched in healthy controls (HLT) as compared to psoriatic arthritis (PsA) patients were detected by LefSe (P<0.05, LDA>2 logs) and after false-discovery rate (FDR). (A) Overall, no taxon was found to be enriched in PsA (red bars) compared to HLT (blue bars) or (B) Ps patients (green bars). Taxa are arranged in descending order according to their LDA score and marked with an asterisk (*) when significance remained after FDR correction. (C) Taxa differentiating PsA from Ps samples. (D) Box-plots with relative abundance (parts per unit) of specific taxa underrepresented in PsA and Ps subjects. Only those with statistical differences after FDR correction are shown.

FIG. 2. Decreased operational taxonomic units (OTUs) in PsA and Ps gut microbiota. Several members of the gut microbial communities are underrepresented in PsA and Ps patients at the OTU level. (A) Bacterial OTUs significantly enriched in healthy controls (blue bars) as compared to PsA (red bars) and (B) Ps patients (green bars) were detected by LefSe. Those significant OTUs with q<0.2 after false-discovery rate (FDR) analysis are indicated by asterisks (*). (C) OTUs with distinct abundance in PsA and Ps. (D) Box-plots with relative abundance (parts per unit) of those OTUs marked with * in A-C.

FIG. 3. Gut lumen and serum sIgA, RANKL, OPG and S100 levels. (A) Levels of gut lumen sIgA are higher in PsA patients compared to controls, while measurements in serum revealed no differences among groups. (B) Fecal RANKL were not measurable in most PsA patients, while serum levels were similar among groups. (C) Levels of OPG in fecal samples were higher in Ps vs controls, but similar to those in PsA. (D) Serum S100 levels were significantly higher in Ps compared to PsA and controls. Note: Fecal samples were not available for fecal measurements for two healthy subjects. Fecal RANKL, OPG and S100 were not available in one PsA patient.

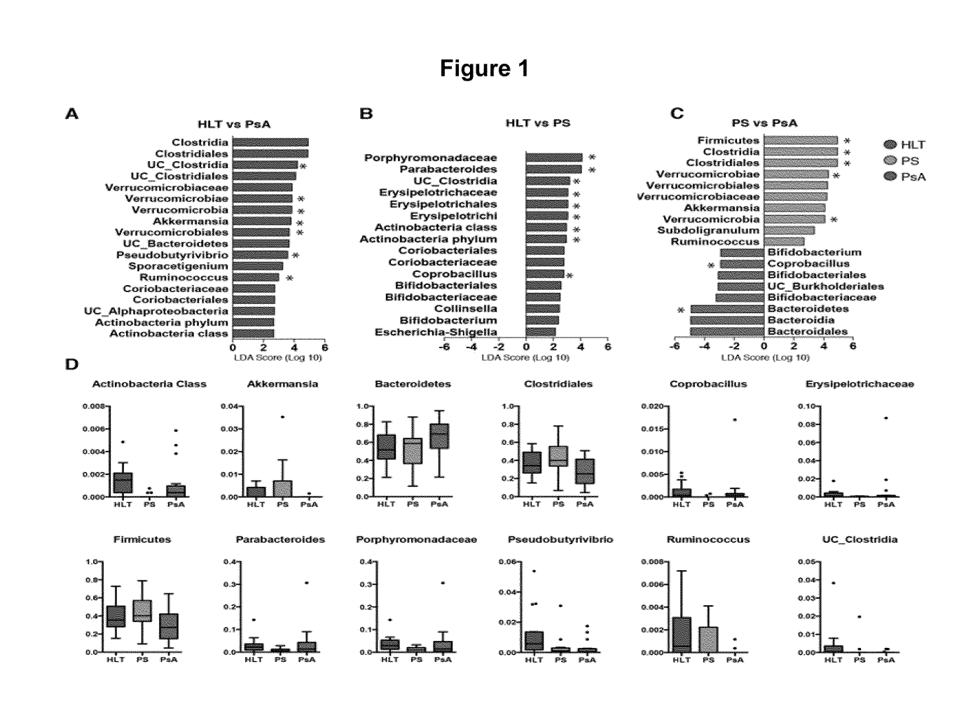

Supplementary FIG. 1. Phylogenetic diversity among groups. There is an overall decrease in gut microbiota diversity in psoriatic arthritis (PsA) and psoriasis of the skin (Ps) compared to healthy controls (HLT) as calculated by the Shannon diversity index (A; P<0.05) and Faith's phylodiversity index (B; P<0.05). Unweighted UniFrac analysis reveals a significant clustering between groups based on their overall microbiota composition (C; P<0.05).

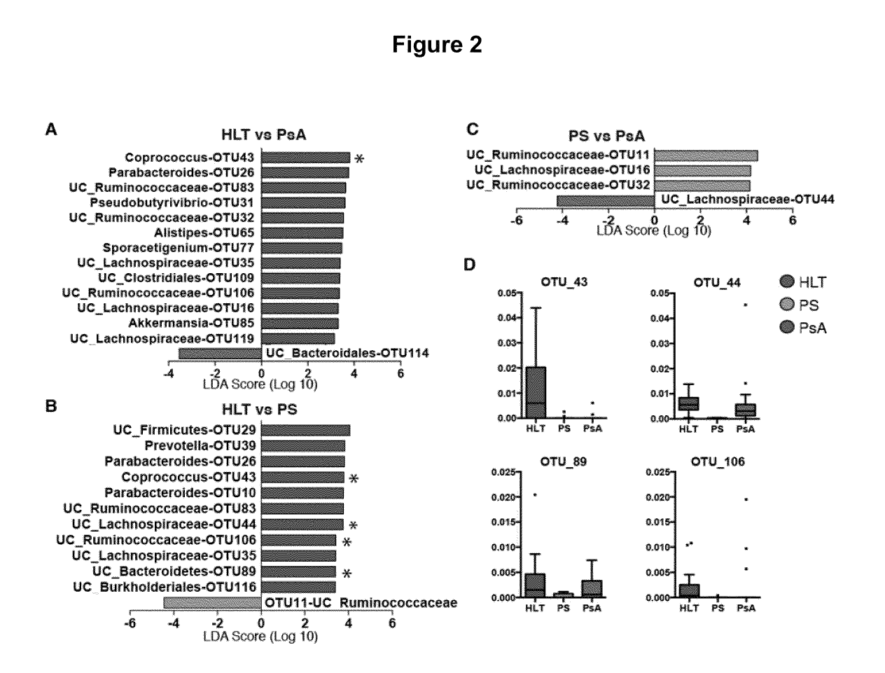

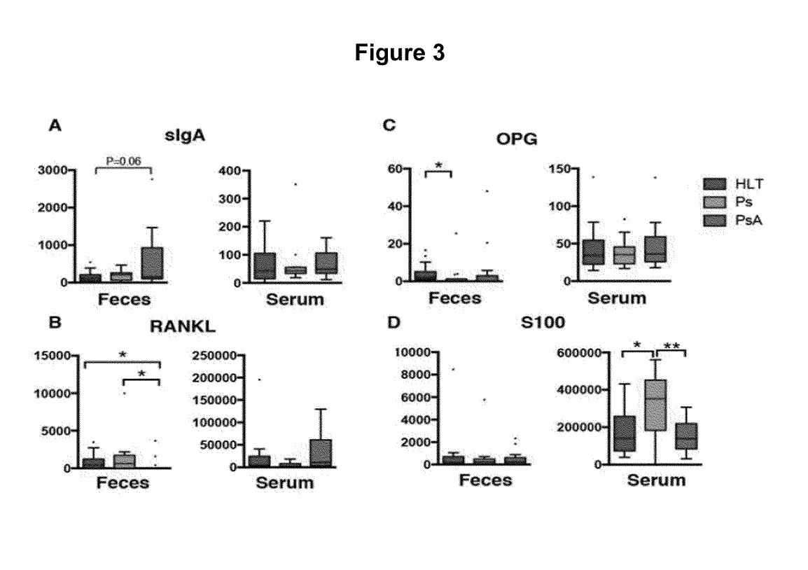

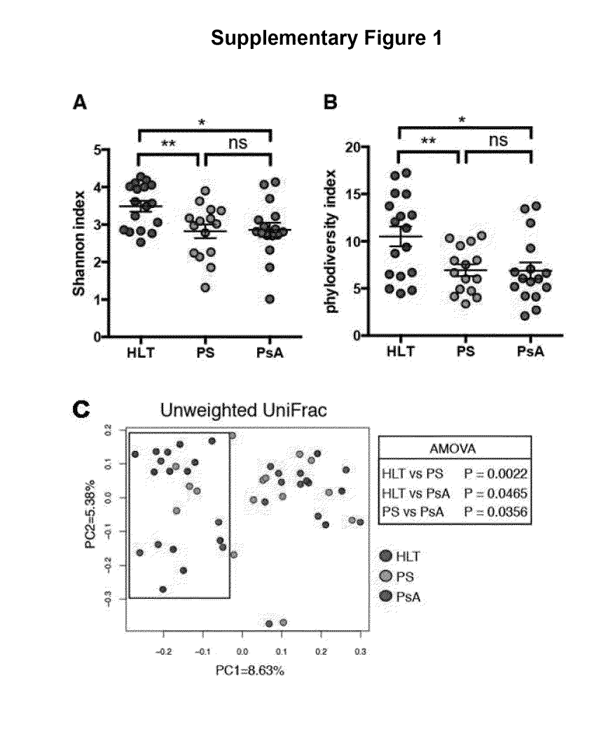

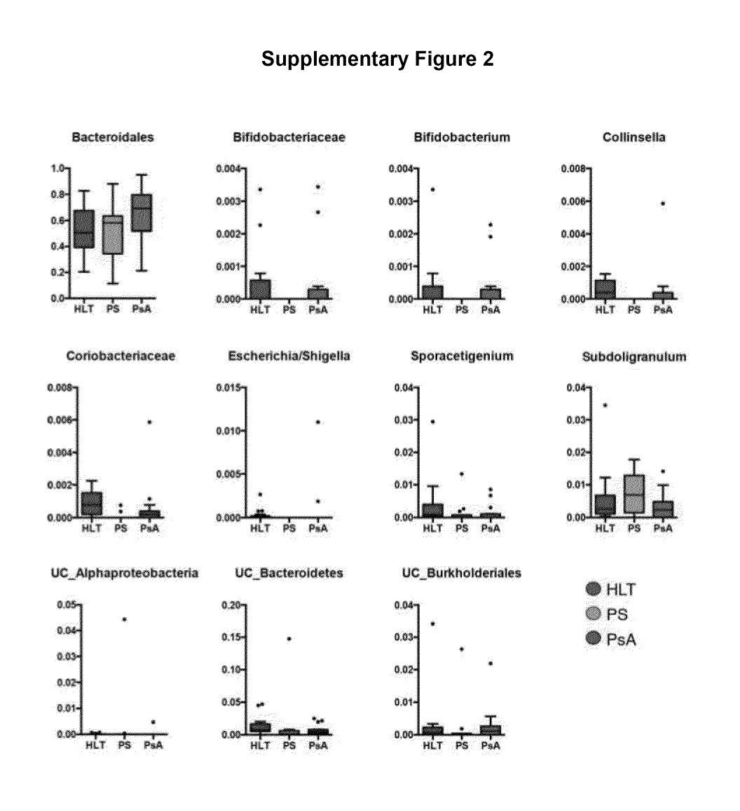

Supplementary FIG. 2. Taxa different by LefSe.

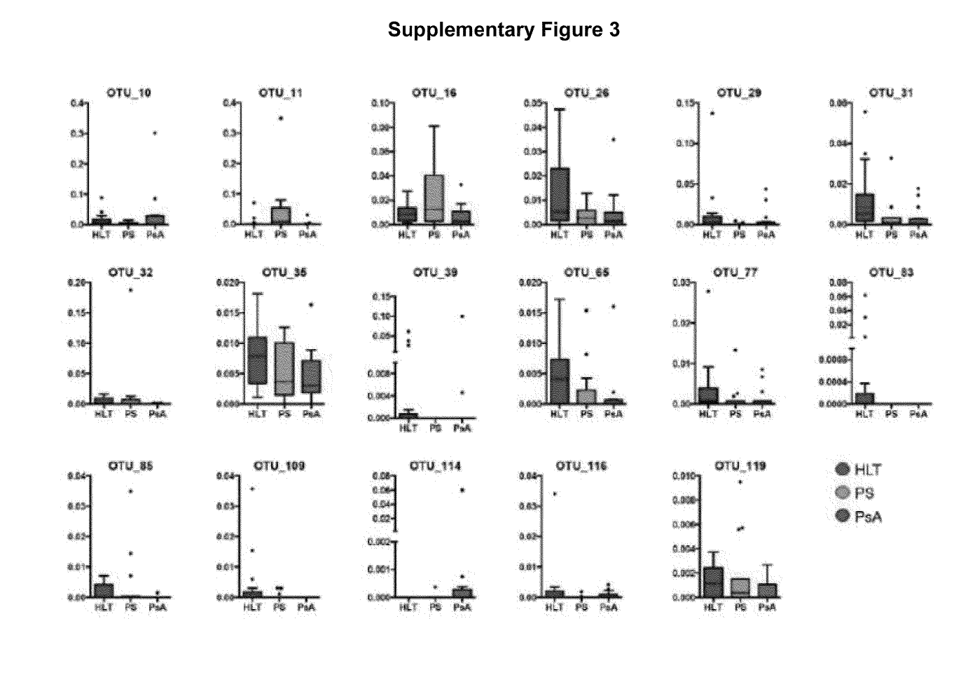

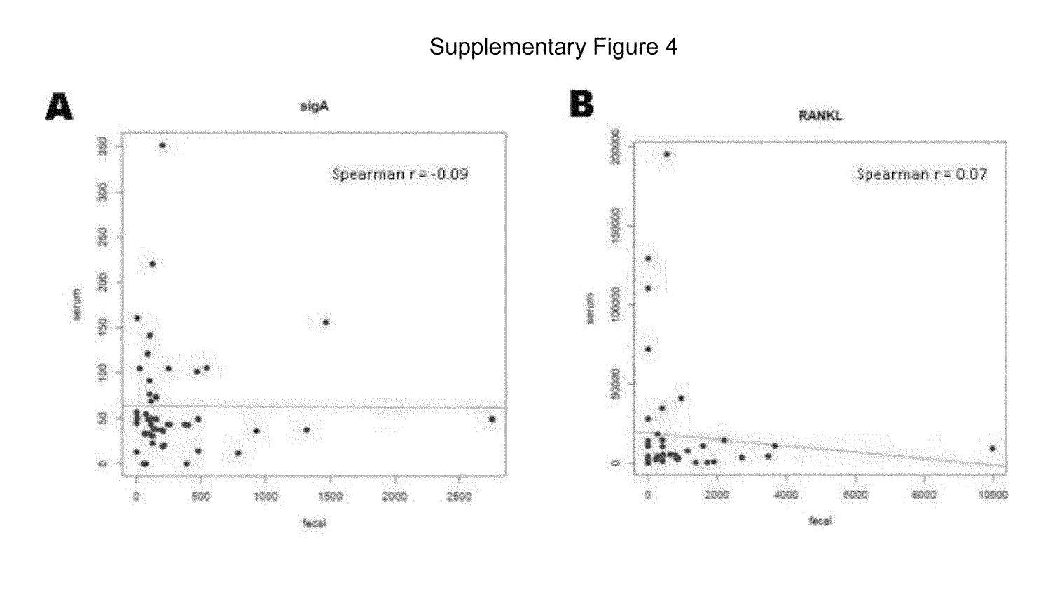

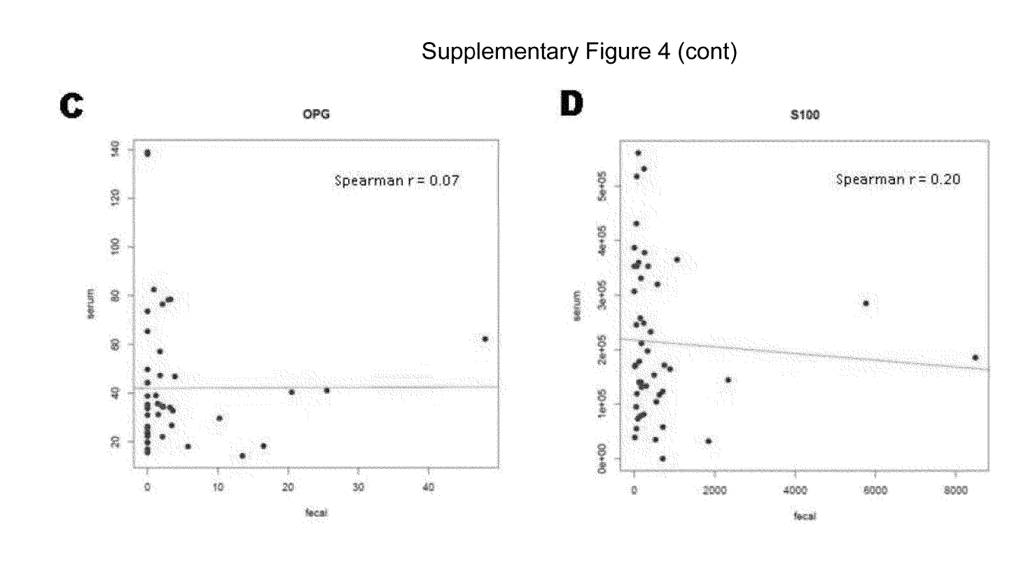

Supplementary FIG. 3. OTUs different by LefSe.

Supplementary FIG. 4. Correlation coefficient between serum and fecal samples. Correlation coefficient between serum and fecal levels revealed no statistical difference when analyzing (A) sIgA, (B) RANKL, (C) OPG, and (D) S100.

### DETAILED DESCRIPTION

Psoriatic arthritis (PsA) is a type of chronic spondyloarthritis (SpA), typically affecting individuals with pre-existing psoriasis of the skin (Ps). Despite recent advances in diagnosis and treatment, the pathogenesis of PsA remains unclear. The prevalent paradigm posits that in the presence of predisposing genetic factors (e.g., HLA-B*27, Cw6), individuals with Ps will develop PsA after exposure to as yet unidentified environmental factors (1,2). Previously proposed triggers include viruses, bacterial infections, trauma and stress. Interest has recently reemerged concerning the role of the gut microbiome (the totality of bacteria and their genes in a given biological niche) and associated gut inflammation in the pathogenesis of the SpA disease spectrum (3-6). HLA-B27 over-expressing rats, for instance, develop arthritis and colitis only in the presence of specific intestinal microbes (7). Similarly, SKG mice develop joint inflammation, enthesitis, skin inflammation and ileitis after injection with β-glucan, a major component of bacterial and fungal cell walls (8).

Strong epidemiologic evidence also suggests an intimate relationship between intestinal and joint inflammation in SpA. Patients with PsA, Ps and ankylosing spondylitis (AS) experience a much higher incidence of inflammatory bowel disease (IBD). Furthermore, articular manifestations are found in more than one-third of patients with known Crohn's disease or ulcerative colitis (UC) (9). This has led to the consideration of IBD-related arthritis as part of the SpA spectrum.

Several studies have further associated intestinal mucosal inflammation and human SpA. Approximately 70% of all SpA patients show at least some ileocolonoscopic or histologic alterations in the intestinal epithelium. PsA has specifically been shown to be associated with both subclinical gut inflammation (10) and a significantly increased risk of subsequent Crohn's disease (11).

Therefore, several animal and human studies substantiate the hypothesis pointing toward a biological link between (local) gut and (systemic) joint inflammation. This suggests a common etiology, but its precise nature remains unknown.

Among sites of exposure to bacterial antigens, the intestinal mucosa represents a unique environment for triggering of local and distal autoimmunity. The human intestinal microbiome contains roughly 100 trillion cells whose genomes encode ˜3.3 million protein-coding genes (100-fold more than the human genome). The NIH Human Microbiome Project was recently launched to better understand and define this collective human-microbiome “supraorganism” in both health and disease.

Utilizing novel high-throughput DNA sequencing, it is now possible to identify bacteria in a given community, including unculturable or fastidious organisms, without the need for conventional microbiology techniques. The present study aimed to describe, for the first time, potential alterations in gut microbiota composition of patients with PsA and associated local inflammatory response, compared to Ps and healthy controls.

At the outset, the present inventors set out to characterize the abundance and diversity of gut microbiota in patients with never-treated, new-onset psoriatic arthritis (PsA). As described in detail herein, high-throughput 16S rRNA pyrosequencing was utilized to compare the community composition of gut microbiota in PsA patients, Ps patients, and healthy, matched controls. Samples from patients with PsA, psoriasis of the skin (Ps), new-onset rheumatoid arthritis (NORA) and healthy controls were also assessed for the presence and levels of fecal secretory immunoglobulin A (sIgA) and various proteins and pro-inflammatory cytokines.

As described herein, a total of 48 fecal samples were obtained from PsA, Ps and healthy subjects for sequencing. Using a distance-based similarity of ≧97% for operational taxonomic units (OTU) assignment, a total of 2835 OTUs were identified. As shown in Supplementary FIGS. 1A and B, microbial diversity was significantly reduced in PsA and Ps samples when compared to healthy subjects, as calculated by the Shannon diversity index and Faith's phylodiversity index. The present inventors also assessed whether the overall structure of the microbiota of healthy samples differed from that of Ps and PsA and quantified the similarity by applying the UniFrac phylogenetic distance. PCoA was further applied to cluster samples along orthogonal axes of maximal variance. As shown in Supplementary FIG. 1C, PC1 axes discriminated most healthy samples from the majority of Ps and PsA samples. Analysis of molecular variance (AMOVA) of the obtained UniFrac distances between samples revealed that overall microbiota structure was also significantly different when comparing PsA to Ps samples (Supplementary FIG. 1).

To investigate further which bacterial taxa were distinct among groups, LefSe analysis was applied (see Methods). Interestingly, while no bacterial taxa were found to be enriched in PsA patients, relative abundance of several microbial clades was decreased in both PsA and Ps, and therefore enriched in healthy controls (FIG. 1 and Supplementary FIG. 2). Within these identified components of the intestinal microbiota, Akkermansia, Ruminococcus and Pseudobutyrivibrio were considered the most relevant genera that discriminated PsA microbiota from healthy controls (FIG. 1A, D). At other levels of taxonomic classification, unclassified Clostridia and the parental taxonomic levels of Akkermansia (Verrucomicrobia, Verrucomicrobiae and Verrucomicrobiales) were also significantly decreased in PsA. The Ps gut microbiota was characterized by a reduced relative abundance of the genera Parabacteroides and Coprobacillus (FIG. 1B, D). The comparison between PsA and Ps groups revealed that the higher taxonomic levels for Akkermansia and Ruminoccocus (including Firmicutes/Clostridiales and Verrucomicrobiales, respectively) were significantly less abundant in PsA patients (FIG. 1C, D), while Bacteroidetes phylum and Coprobacillus genus were less abundant in Ps samples. Akkermansia and Ruminoccocus per se were also relatively decreased in PsA (FIG. 1C, Supplementary FIG. 2).

Analysis of the microbiota beyond the genus level was undertaken to investigate the various OTUs that were underrepresented in patients with PsA. Several OTUs had a decreased relative abundance compared to healthy controls, including OTUs 43 (Coprococcus), 31 (Pseudobutyrivibrio), 26 (Parabacteroides), 83 (unclassified_{—}Ruminococcaceae), 65 (Alistipes), and 85 (Akkermansia) (FIG. 2A, 2D and Supplementary FIG. 3). Intriguingly, several of these OTUs, including OTU43 as well as OTUs 26, 83, and 35 were also decreased in Ps patients, suggesting a possible common gut microbiota signature for Ps and PsA (FIG. 2 and Supplementary FIG. 3). Moreover, Ps samples showed a significantly decreased relative abundance of several other OTUs when compared to healthy subjects, including OTUs 44, 89 and 106 (FIG. 2B, 2D). OTU11 was the only overrepresented OTU in the Ps group. When comparing PsA and Ps groups, patients with skin disease only had increased relative abundance of OTUs 11, 16 and 32, while patients with PsA had an overrepresentation of OTU44 only (FIG. 2C, Supplementary FIG. 3).

Luminal concentrations of sIgA were measured in all groups to investigate further whether this alteration in intestinal microbial communities in PsA or Ps patients was associated with a differential local immune response. Given their proposed role in PsA pathogenesis (18), fecal levels of RANKL and OPG were also measured, along with 5100 (a novel neutrophil-derived mucosal marker for IBD). PsA patients had an increase in sIgA relative to healthy controls (FIG. 3A; P=0.06). Conversely, fecal RANKL was significantly reduced in PsA (FIG. 3B; P<0.05), with only 19% of patients having measurable RANKL compared to 30% of healthy subjects and 75% of Ps patients. Fecal OPG was significantly lower in Ps vs controls (FIG. 3C; P<0.05) but not when compared to PsA. Levels of fecal S100 were similar among all groups. To determine whether fecal quantities of all these proteins were a reflection of systemic levels (which may have translocated into the gut lumen), concomitant serum measurements were performed to calculate correlation coefficients. Although there was no significant correlation between fecal and serum levels of these proteins (Supplementary FIG. 4), serum levels of S100 were significantly elevated in Ps patients compared to PsA and healthy subjects (FIG. 3D; P<0.05).

Because of univariate associations between groups, gut microbiota and metadata, the present inventors set out to describe correlations between decreased taxa in Ps and PsA and the various measured fecal and serum proteins. An optimal Bayesian network, which incorporates correlations between taxa, was also performed. Akkermansia (as well as OTU85) was inversely correlated with fecal levels of sIgA. Interestingly, Coprobacillus, a genus decreased in Ps, negatively correlated with 5100 levels in serum. OTU43 (Coprococcus) correlated with OTU31, which in turn positively correlated with OTU109, all of which were also decreased in both groups of patients. OTU16 (UC_{—}Lachnospiraceae), low in PsA, was the only OTU positively correlated with fecal levels of RANKL (also decreased in PsA), and co-occurred with two other OTUs that were relatively decreased in PsA: OTU32 and OTU119. Taken together, these interactions describe a potential distinctive pattern representative of the PsA gut microbiota, characterized by lower relative abundance of several taxa and decreased levels of fecal RANKL.

Bayesian network analysis, furthermore, showed that OTUs 10 (Parabacteroides), 44 and 35 (both UC_{—}Lachnospiraceae) also clustered together, with the latter taxa revealing the highest inverse correlation with serum levels of 5100. These interactions differentiate the gut microbiota of the Ps cohort and its associations with systemic inflammatory markers.

Results presented herein, therefore, reveal that PsA and Ps patients have a lower relative abundance of multiple intestinal bacteria. Although some genera were concomitantly decreased in both conditions, PsA samples had lower abundance of reportedly beneficial taxa. This gut microbiota profile in PsA was, moreover, associated with changes in specific inflammatory proteins unique to this group, and distinct from Ps and controls.

Further to the above, the present inventors have demonstrated that patients with untreated PsA have a low prevalence/abundance of multiple intestinal bacteria at disease onset. This gut microbiota profile in patients with recent onset PsA was similar to, but distinct from, that described for patients with IBD and markedly different from that of patients with new-onset RA. Although levels of fecal sIgA are increased in most cases of inflammatory arthritis, a low level of RANKL in fecal samples is a hallmark of PsA patients.

It is, moreover, noteworthy that although some genetic and environmental features appear to be shared among disorders characterized within the spectrum of spondyloarthropathies, many therapeutic approaches are specific to disease phenotype. Etanercept (a TNF-α inhibitor), for example, used for the treatment of Ps and PsA, was supposed to be effective in Crohn's and IBD. However, clinical trials failed to show significant improvement in those patients and etanercept was never approved for this indication by the Food and Drug Administration (FDA). Similarly, the new biologic therapies, including usetikinumab (an IL-12/23 blocker) and apremilast (a PDE4 inhibitor), are specifically prescribed for Ps and PsA, but not for IBD, ankylosing spondylitis or reactive arthritis. These, and other examples, are proof that the various disease phenotypes have different pathogenetic pathways that can be targeted through a variety of mechanisms and compounds. Using therapeutic approaches specific to a particular disease phenotype may also benefit from greater pharmacologic specificity, which is generally associated with fewer side effects.

In accordance with these discoveries, methods for treating Ps and/or PsA are presented herein that comprise administering to the subject afflicted with Ps and/or PsA a therapeutically effective amount of a sample comprising a fecal transplant isolated from a healthy donor or a therapeutically effective amount of a composition comprising at least one purified bacterial species of a genera of Akkermansia, Ruminococcus, Pseudobutyrivibrio, and Coprococcus, wherein administering the therapeutically effective amount of the fecal transplant or the at least one purified bacterial species treats the subject afflicted with Ps and/or PsA. Also encompassed herein is a fecal transplant isolated from a healthy donor or at least one purified bacterial species of a genera of Akkermansia, Ruminococcus, Pseudobutyrivibrio, and Coprococcus or a composition thereof for use in the treatment of Ps and/or PsA in a subject in need thereof. Use of a fecal transplant isolated from a healthy donor or at least one purified bacterial species of a genera of Akkermansia, Ruminococcus, Pseudobutyrivibrio, and Coprococcus or a composition thereof in the preparation of a medicament for the treatment of Ps and/or PsA is also envisioned herein.

Fecal transplant or fecal microbiota transplantation (FMT) has been used successfully for the treatment of Clostridium difficile infections (CDI), including therapy resistant forms thereof. As described in Borody et al. (Curr Gastroenterol Rep. 15: 337, 2013; the entire content of which is incorporated herein by reference) and understood in the art, FMT material is derived from healthy donors who have no risk factors for transmissible diseases and have not been exposed to agents, such as, for example, antibiotics, that could alter the composition of their gut microbiota. FMT donor selection criteria and screening tests are outlined in detail in published international guidelines established by the FMT Working Group (Bakken et al. Clin Gastroenterol Hepatol. 9:1044-9, 2011). Details pertaining to the harvesting and processing of FMT material are known in the art and are reviewed in Borody et al. (supra). Briefly, many protocols call for use of fresh feces, which requires collection and processing on the same day scheduled for the FMT. Other protocols have been developed that use highly filtered human microbiota mixed with a cryoprotectant, which can be frozen for storage at −80° C. until required for use (Hamilton et al. Am J Gastroenterol. 107(5):761-7, 2012). This approach benefits from convenience with regard to scheduling, and generates a processed fecal material (fecal filtrate) having reduced volume and fecal aroma. Equivalent clinical efficacy has been noted when either purified processed fecal material or fresh, partly filtered feces were used in CDI. FMT material may be administered via naso-duodenal, transcolonoscopic, or enema based routes.

With respect to the generation and administration of isolated, purified populations of at least one bacterial species of a genera of Akkermansia, Ruminococcus, Pseudobutyrivibrio, and Coprococcus or combinations of isolated, purified populations thereof and compositions comprising same, a number of bacterial species are commercially available. Such species include: the Coprococcus species designated ATCC®29549™ (strain designation PE15; which was isolated from rumen and is known to degrade phloroglucinol); ATCC®27761™; ATCC®27758™; or ATCC®27759™; the Ruminococcus species Ruminococcus gnavus (ATCCBAA-35913™; strain designation AB[VI-268]; isolated from human feces) or Ruminococcus gnavus (ATCC29149™; strain designation VPI C7-9; isolated from human feces); strain designation L1-82; isolated from human infant feces); the Akkermansia species Akkermansia muciniphila (ATCCBAA-835™; the Pseudobutyrivibrio species designated ATCC® BAA-455™; the Coprobacillus species Coprobacillus cateniformis (DSMZ No. 15921), and strain designation Muc[CIP 107961], which is isolated from human feces and is known to degrade mucin. Culturing conditions for these bacterial species are listed on the relevant product information and are, moreover, known in the art. Additional information pertaining to culturing conditions and methods for preparing suitable populations of isolated, purified populations of bacterial species or combinations thereof, as well as compositions of these bacterial populations and dosing regimens is presented in U.S. Pat. No. 8,092,793, the entire content of which is incorporated herein in its entirety.

Additional species suitable for use in accordance with methods and in compositions detailed herein are described in Holdeman et al. (1974, Int J Systematic Bacteriology 24:260-277), the entire content of which is incorporated herein by reference.

In a further aspect, diagnostic biomarkers/indicators described herein are also envisioned as therapeutic biomarkers/indicators. In that determining the presence and/or amount of one of the aforementioned biomarkers/indicators (e.g., a bacterial genus or species that is underrepresented in PsA and/or Ps) can be used for diagnosing PsA and/or Ps and/or for predicting the likelihood that a subject will be afflicted with PsA and/or Ps, it is envisioned that determining the presence and/or amount of one of these biomarkers/indicators can also be used as a therapeutic indicator. It is to be understood that in such therapeutic embodiments, detection of the relevant biomarkers/indicators is performed before and after administration of the potential therapeutic compound for the purposes of comparison.

In a particular embodiment, detection of the presence of or an increase in bacteria of at least one of the following bacterial genera: Akkermansia, Ruminococcus, Pseudobutyrivibrio, and Coprococcus or a particular species thereof, such as R. gnavus, following treatment with a potential therapeutic compound (e.g., a biotic supplement such as a composition described herein or a fecal sample) would indicate that the therapeutic compound is efficacious. Under such a circumstance, the presence of or an increase in a species of the Coprococcus genera, for example, following treatment as compared relative to the low or negligible levels these species prior to treatment indicates that the compound is efficacious.

In another particular embodiment, detection of no change in the levels of or a decrease in bacteria of at least one of the following bacterial genera: Akkermansia, Ruminococcus, Pseudobutyrivibrio, and Coprococcus or a particular species thereof, such as R. gnavus, following treatment with a potential therapeutic compound would indicate that the therapeutic compound is not efficacious. Under such a circumstance, the absence of a change in levels of or an even further decrease in a species of the Coprococcus genera, for example, following treatment as compared relative to the low or negligible levels of these species prior to treatment indicates that the compound is not efficacious.

The identification of biomarkers/indicators (e.g., underrepresentation of a bacterial genus or species associated with or indicative of PsA and/or Ps) for assessing risk for PsA and/or Ps, or for diagnosing PsA and/or Ps as set forth herein and methods of using same makes available a straightforward assay whereby a stool sample can be used to identify subjects/patients at-risk for PsA and/or Ps development and/or in the early phases of disease, so therapy can be instituted and tissue damage, deformity and disability that are particularly relevant with regard to PsA can potentially be prevented. Diagnosing PsA can be challenging for a variety of reasons, not the least of which is the observation that some patients present with tendinitis, but no frank arthritis. Absent a definitive diagnosis, such patients may remain under-diagnosed or undiagnosed for years, during which time they are not properly treated, thereby permitting the disease to progress to the point where the PsA is advanced and affects the joints. Various methods described herein and known in the art can be utilized to assess biomarkers/indicators identified herein, including, without limitation, nucleic acid sequencing and antibodies specific for the indicated bacterial taxa and/or species.

There is a need for improved methods for determining PsA risk, particularly in those patients with a familial history of Ps and/or PsA. There is, moreover, a need for diagnostic tools with which skilled practitioners can monitor asymptomatic, high risk patients using minimally invasive techniques to assess, on an ongoing basis, risk of PsA onset. Improved diagnostic tools with which skilled practitioners can determine how best to treat a patient diagnosed with PsA are also sought. These tools can, furthermore, be applied to methods for assessing if a therapeutic regimen is efficacious for the patient. The discoveries described herein address the above-indicated long sought diagnostic, prognostic, and therapeutic needs.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The subject or patient is preferably an animal, including but not limited to animals such as mice, rats, cows, pigs, horses, chickens, cats, dogs, etc., and is preferably a mammal, more preferably a primate, and most preferably a human.

In a particular embodiment, the animal is of an animal model species, for example, a mouse animal model system of Ps and/or PsA. Such animal models are known in the art and described in, for example, Weitz et al. (Curr Rheumatol Rep. 2013 November; 15(11):377; the entire content of which is incorporated herein by reference). Animal models described therein and in references cited in Weitz et al., each of which references is incorporated herein in its entirety, include, for example: the mouse collagen-induced arthritis model, the tumor necrosis factor (TNF) tg model, HLA-B27/B2 transgenic rat and beta 2 micro globulin knockout mouse, beta glucan-ZAP 70 mice, JunB/c-jun and amphiregulin, and the DBA/1 mouse and MHC II mouse model.

The term “preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention” and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term psoriatic arthritis or PsA refers to a disease/condition in an individual that meets three or more points from the ClASsification of Psoriatic ARthritis (CASPAR) criteria, including: (1) the presence of psoriasis (current, history of, or family history of), (2) psoriatic nail dystrophy, (3) a negative rheumatoid factor (RF) test result, (4) dactylitis (history of or current), and (5) radiographic evidence of juxta-articular new bone formation.

As used herein, the term psoriasis or Ps refers to a disease/condition characterized by patches of thick, inflamed skin covered with silvery scales. These patches, or plaques, are usually itchy. They most often occur on the elbows, knees, other parts of the legs, scalp, lower back, face, palms, and soles of the feet, but they can occur on skin anywhere on the body. The disease may also affect the fingernails, the toenails, and the soft tissues of the genitals, and inside the mouth.

As used herein, the term arthritic symptoms refers to joint swelling, tenderness, redness, warmth or a combination thereof.

As used herein, the term fecal transplant refers to fecal bacteria isolated from a healthy individual and thereby processed by the hand of man, which is transplanted into a recipient. In a particular embodiment, the fecal transplant is manmade processed fecal material (fecal filtrate) having reduced volume and/or fecal aroma relative to unprocessed fecal material. In a more particular embodiment, the fecal transplant is a fecal bacterial sample. The term fecal transplant may also be used to refer to the process of transplantation of fecal bacteria isolated from a healthy individual into a recipient. It is also referred to as fecal microbiota transplantation (FMT), stool transplant or bacteriotherapy.

As used herein, the term fecal bacterial sample refers to an essentially pure or purified bacterial population (e.g., a paste thereof), which is purified from feces and thus, is essentially free of fibrous fecal material that is normally associated with feces in a natural state prior to manipulation by the hand of man.

As used herein, the term healthy donor refers to individuals without history of any chronic medical condition.

As used herein, the term pure or purified bacterial species refers to a monoculture of a single bacterial species that consists essentially of or consists of only bacterial cells of the indicated species. In other words, a pure or purified bacterial species is essentially devoid of bacterial cells of other different bacterial species.

As used herein, the term analyzing microbiota diversity refers to assessing the presence and/or absence of commensal bacterial species in a sample (e.g., a fecal sample).

As used herein, the term microbiome profile refers to the relative abundance of commensal bacterial species identified in a fecal sample.

As used herein, the term pathogenic microbial dysbiosis refers to a state/condition in which the relative abundance of commensal bacterial species differs statistically from that observed in a healthy state/condition.

As used herein, the term “immune response” signifies any reaction produced by an antigen, such as a protein antigen, in a host having a functioning immune system Immune responses may be either humoral, involving production of immunoglobulins or antibodies, or cellular, involving various types of B and T lymphocytes, dendritic cells, macrophages, antigen presenting cells and the like, or both Immune responses may also involve the production or elaboration of various effector molecules such as cytokines, lymphokines and the like Immune responses may be measured both in in vitro and in various cellular or animal systems.

An “immunological response” to a composition or vaccine comprised of an antigen is the development in the host of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

The phrase “pharmaceutically acceptable” refers to molecular entities and compositions that are physiologically tolerable and do not typically produce an allergic or similar untoward reaction, such as gastric upset, dizziness and the like, when administered to a human. The phrase “physiologically compatible” refers to molecular entities and compositions that do not alter to a significant degree biological processes.

The phrase “therapeutically effective amount” is used herein to mean an amount sufficient to preferably reduce by at least about 30 percent, more preferably by at least 50 percent, most preferably by at least 90 percent, a clinically significant change in a pathological feature of a disease or condition. In an embodiment thereof, a therapeutically effective amount of a bacterial population, including an essentially pure or purified population of a particular bacterial species or a combination of at least two essentially pure or purified populations of bacterial species, is 10⁶ to 10¹² colony forming units (cfu). Such a therapeutically effective amount may be administered or used at a concentration of 10⁶ to 10¹² cfu/ml and at a dose of 1 ml/100 g body weight for a small animal, such as a mouse or rat. In a further embodiment thereof, 10⁸ cfu/ml is administered or used at a dose of 1 ml/100 g body weight for a small animal Such dosing parameters can be scaled up for larger animals, including humans, using standard means known in the art.

Compositions containing molecules or compounds described herein can be administered for diagnostic and/or therapeutic treatments. In therapeutic applications, compositions are administered to a patient already suffering from Ps and/or PsA in an amount sufficient to at least partially arrest the symptoms of the disease and its complications. An amount adequate to accomplish this is defined as a “therapeutically effective amount or dose.” Amounts effective for this use will depend on the severity of the disease and the weight and general state of the patient.

Compounds, such as antibiotics (e.g., vancomycin), for use in treating PsA may be prepared in pharmaceutical compositions, with a suitable carrier and at a strength effective for administration by various means to a patient experiencing an adverse medical condition associated with PsA, wherein a decrease in aforementioned bacterial genera or species has been detected, for the treatment thereof. In exemplary fashion, a PsA patient exhibiting microbial dysbiosis as described herein, could be treated with vancomycin or another suitable antibiotic in advance of treatment with a composition described herein or a fecal sample. A skilled practitioner could, moreover, choose an antibiotic based on its activity spectrum so as to minimize the effect of the antibiotic on beneficial bacterial genera and/or species that are associated with a healthy state in subjects. A variety of administrative techniques may be utilized, among them parenteral techniques such as subcutaneous, intravenous and intraperitoneal injections, catheterizations and the like. Average quantities of the compounds or derivatives thereof may vary and in particular should be based upon the recommendations and prescription of a qualified physician or veterinarian.

Also encompassed herein are therapeutic compositions useful for practicing the therapeutic methods described herein. A subject therapeutic composition may include, in admixture, a sterile manmade pharmaceutically acceptable or physiologically compatible excipient (carrier) and one or more of an agent as an active ingredient (e.g., a purified bacterial species or at least one purified bacterial species or a byproduct of a bacterial genera or species, underrepresentation of which is correlated herein with PsA and/or Ps, and/or risk thereof), which promotes normalization or restoration of normal immune response in the gut and systemically so as to reduce inflammatory immune responses, as described herein. Compositions wherein the active ingredient is a purified bacterial species (monoculture) or at least one purified bacterial species comprise, in addition to the active ingredient, a sterile manmade pharmaceutically acceptable or physiologically compatible excipient or carrier. In accordance with same, the purity of the composition with regard to the bacterial content is maintained and preserved.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or cell modulation desired. Precise amounts of active ingredient required to be administered depend on the judgment of the practitioner and are peculiar to each individual. However, suitable dosages may range from about 0.1 to 20, preferably about 0.5 to about 10, and more preferably one to several milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

As used herein, the term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. In a particular embodiment, the isolated nucleic acid sequence is a cDNA.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The terms “percent similarity”, “percent identity” and “percent homology” when referring to a particular sequence are used as set forth in the University of Wisconsin GCG software program and are known in the art.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence. When referring to a composition comprising a bacterial species, for example, the composition may consist essentially of the bacterial species, thereby indicating the population of the bacterial species in the composition is present in the absence of other bacterial species or exists as a purified or pure population (monoclulture) of the bacterial species in question. When referring to a composition consisting essentially of or consisting of more than one purified population of a bacterial species, the mixture of bacterial species monocultures that were combined in the composition exists in the absence of other bacterial species. By way of example, a composition that consists essentially of bacterial species A, B, and C does not include any bacterial cells of other (non-A, -B, or -C) bacterial species.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, which facilitate the expression of a polypeptide coding sequence in a host cell or organism.

As used herein, the term “operably linked” refers to a regulatory sequence capable of mediating the expression of a coding sequence, which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to a primer and a probe as described herein and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers and/or probes may be labeled fluorescently with 6-carboxyfluorescein (6-FAM). Alternatively primers may be labeled with 4, 7, 2′,7′-Tetrachloro-6-carboxyfluorescein (TET). Other alternative DNA labeling methods are known in the art and are contemplated to be within the scope of the invention.

In a particular embodiment, oligonucleotides that hybridize to nucleic acid sequences identified as specific for, for example, any one of a bacterial genera or species underrepresented in PsA and/or Ps as described herein, are at least about 10 nucleotides in length, more preferably at least 15 nucleotides in length, more preferably at least about 20 nucleotides in length. Further to the above, fragments of nucleic acid sequences identified as specific for a bacterial genera or species underrepresented in PsA and/or Ps described herein represent aspects of the present invention. Such fragments and oligonucleotides specific for same may be used as primers or probes to determining the amount of a bacterial genera or species underrepresented in PsA and/or Ps in a biological sample obtained from a subject. Primers such as those described herein, which bind specifically to a bacterial genera or species underrepresented in PsA and/or Ps may, moreover, be used in polymerase chain reaction (PCR) assays in methods directed to determining the amount of a bacterial genera or species underrepresented in PsA and/or Ps in a biological sample obtained from a subject.

By “solid phase support or carrier” is intended any support capable of binding an oligonucleotide, antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present methods and/or compositions. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art are aware of many other suitable carriers for binding oligonucleotide, antibody, or antigen, and are able to ascertain the same by use of routine experimentation.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contain the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

### Kits

Also encompassed herein is a diagnostic pack or kit comprising one or more containers filled with one or more of the diagnostic reagents described herein. Such diagnostic reagents include fragments and oligonucleotides useful in the detection of a bacterial genera or species underrepresented in PsA and/or Ps in a subject or sample isolated therefrom. Diagnostic reagents may comprise a moiety that facilitates detection and/or visualization. Diagnostic reagents may be supplied in solution or immobilized onto a solid phase support. Optionally associated with such container(s) are buffers for performing assays using the diagnostic reagents described herein, negative and positive controls for such assays, and instructional manuals for performing assays.

Unless defined otherwise, all technical and scientific terms used herein have the same meaning as commonly understood by one of ordinary skill in the art to which this invention belongs.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

All publications mentioned herein are incorporated herein by reference to disclose and describe the methods and/or materials in connection with which the publications are cited.

### Examples

### Materials and Methods

Study Participants.

Consecutive patients from rheumatology clinics and practice offices of New York University School of Medicine were screened for the presence of PsA [samples and sequences obtained from previously described cohort (12)] or psoriasis of the skin (Ps). After informed consent was signed, each patient's medical history and medications were determined A screening musculoskeletal examination, laboratory and radiographic assessments were also performed or reviewed. All PsA/Ps patients who met study criteria were offered enrollment. Non-arthritic healthy subjects were also identified from a recently published study (12) and enrolled as controls.

This study was approved by the Institutional Review Board of NYU School of Medicine, and written informed consent was obtained from all study participants.

Inclusion and Exclusion Criteria.

Patients were included as recent-onset PsA if they met Classification Criteria for Psoriatic Arthritis (CASPAR) including presence of current active psoriasis of the skin (Ps) and arthritis, and had never been treated with systemic disease-modifying anti-rheumatic drugs (DMARDs; oral and/or biologic agents) or steroids. Patients were included as Ps without PsA if they were diagnosed by a dermatologist and had no arthritis, enthesitis or dactylitis (as assessed by a rheumatologist) at enrollment. Healthy controls were age-, sex-, and ethnicity-matched individuals with no personal history of psoriasis, autoimmune disease (including IBD) or inflammatory arthritis. Criteria for inclusion required that all subjects be age 18 years or older.

Exclusion criteria applied to all groups were as follows: recent (<3 months prior) use of any antibiotic therapy, current extreme diet (e.g., parenteral nutrition or macrobiotic diet), known history of malignancy or IBD, current consumption of probiotics, or any gastrointestinal tract surgery leaving permanent residua (e.g., gastrectomy, bariatric surgery, or colectomy).

Sample Collection and DNA Extraction.

Fecal samples were obtained for all participants within 24 hours of production. DNA extraction, amplification of the V1-V2 16S rRNA gene region and 454 pyrosequencing were performed for all samples as recently published (13), the entire content of which is incorporated herein by reference. Briefly, samples were collected and directly suspended in MoBio buffer-containing tubes (MoBio). DNA was extracted within 1 hr of sample collection using a combination of the MoBio Power Soil kit (MoBio) and a mechanical disruption (bead-beater) method as described in Costello et al. (2009 Science 326:1694-1697), the entire content of which is incorporated herein by reference. Samples were stored at −80° C.

V1-V2 16S rRNA Region Amplification and 454/Pyrosequencing.

In brief, for each sample, 3 replicate 25-μl PCRs were performed, each containing 50 ng of purified DNA, 0.2 mM dNTPs, 1.5 mM MgCl2, 1.25 U Platinum Taq DNA polymerase, 2.5 μl of 10×PCR buffer, and 0.2 μM of each primer designed to amplify the V1 and V2 regions as previously described (Turnbaugh et al. Nature 2009; 457:480-4): a modified primer 8F

(5′-CTATGCGCCTTGCCAGCCCGCTCAG-TCAGAGTTTGAT
CCTGGCTCAG-3′; SEQ ID NO: 1),

composite of 454 primer B (underline), linker nucleotides (TC), and the universal bacterial primer 8F (italics); and the modified primer 338R

(5′-CGTATCGCCTCCCTCGCGCCATCAGNNNNNNNNNNNNC
AGCTGCCTCCCGTAGGAGT-3′; SEQ ID NO: 2)

composite of 454 primer A (underline), a unique 12-base barcode (Ns), linker nucleotides (CA), and the broad-range bacterial primer 338R (italics). Replicate PCRs were pooled, and amplicons were purified using the Qiaquick PCR Purification Kit (Qiagen). PCR products were sequenced on a 454 GS FLX Titanium platform following the 454 Roche recommended procedures.

Sequence Analysis.

Sequencing data was compiled and processed using mothur software and converted to standard Fasta format as described in prior studies (13). Briefly, sequences were grouped into operational taxonomic units (OTUs) using the average neighbor algorithm. Sequences with a distance-based similarity of ≧97% were assigned to the same OTU. For each sample, microbial diversity was estimated by calculating the Shannon diversity index or Faith's phylodiversity index. Phylogenetic classification was performed using the Bayesian classifier algorithm, with a boot-strap cutoff value of 60%. For microbiota comparison between samples, only 2718 sequences (number of high-quality sequences obtained from the sample with lowest counts) were used for all data analyses.

For UniFrac analysis, a phylogenetic tree was inferred using clearcut (14), on the 16S rRNA sequence alignment generated by mothur. Unweighted UniFrac was run using the resulting tree. Principal Coordinate of Analysis (PCoA) was performed on the resulting matrix of distances between each pair of samples.

Serum and Fecal Measurement of sIgA, Proteins and Cytokines.

Protein concentration in feces was measured by the BCA method. ELISA assays were performed to determine serum and fecal concentrations of secretory IgA (sIgA; Immundiagnostik AG), receptor activator of nuclear factor kappa-B ligand (RANKL, Immundiagnostik AG), osteoprotegerin (OPG; Raybio), and S-100A12 protein (S100, Circulex) utilizing a validated protocol (15).

Human Leukocyte Antigen (HLA) Allele Determination.

Genomic DNA was isolated from peripheral blood of PsA patients using QIAamp Blood Mini Kit (Qiagen). HLA-B and C alleles were determined by Single Specific Primer-Polymerase Chain Reaction (SSP-PCR) methodologies (Weatherall Institute for Molecular Medicine, Oxford, UK) (12).

Statistical Analysis.

In order to identify differentially abundant bacterial taxa among groups, the present inventors applied the LefSe analytical method (16). In brief, LefSe [linear discriminant analysis (LDA) coupled with effect size measurements] is a metagenomic biomarker-discovery approach based on an algorithm that first performs a nonparametric Kruskal-Wallis test in order to identify bacterial taxa whose relative abundance is significantly different in a group of interest (e.g., PsA) compared to controls (i.e., healthy or Ps). Subsequently, LefSe applies LDA to those bacterial taxa identified as significantly different (P<0.05) and further assesses the effect size of each differentially abundant taxon (16). Only those taxa that obtain a log LDA score >2 are ultimately considered. As a result, LefSe indicates those taxa and OTUs that better discriminate between phenotypes. In addition, since LefSe does not consider multiple hypothesis testing, the present inventors further applied the non-parametric Wilcoxon test for every taxon at every phylogenetic level whose average abundance was higher than 0.1% (in any of the three groups analyzed). The Benjamini and Hochberg false discovery rate test (FDR) was then applied. Those bacterial taxa/OTUs with P<0.05 and FDR q value lower than 0.2 were considered as the main bacterial taxa differentiating between groups of samples. For cross-sectional analyses of baseline characteristics and comparison of diversity indexes between groups, differences were evaluated using Student's t-test, Mann-Whitney U test, one-way ANOVA, AMOVA or chi-square test, as appropriate. Two-tailed tests were used for significance testing, and P values less than 0.05 were considered significant.

Correlation and Network Analyses.

Spearman correlation between taxa/OTUs, FAs, sIgA and inflammatory proteins was performed using the statistical R package command cor.test. Correlations were performed only in those taxa/OTUs found to be statistically significant between groups by LefSe. P values under 0.05 were considered significant.

The Optimal Bayesian network structure was inferred through ‘high climbing’ algorithm implemented in the bnlearn R package (17). Regularized inference was carried out by rejecting those relations between nodes with an associated Spearman correlation p-value greater than 0.05.

### Results

Patients.

Only patients with recent-onset, DMARD-naive PsA were included in the study; 56% were female and mean age was 46.2 years (Table 1). Mean disease duration was 0.8 months and no patient had ever received steroids, oral DMARDs, or biologic drugs. All patients had active skin psoriatic lesions and clinical or radiographic evidence of arthritis at enrollment (25% presented with axial arthritis). Ps and healthy controls were age-, sex-, and ethnicity-matched to PsA subjects. Baseline characteristics are described in Table 1.

TABLE 1
Demographic and clinical data among patients with recent-onset psoriatic
arthritis (PsA), psoriasis of the skin (Ps) and healthy control participants.

| | | | Healthy |
|---|---|---|---|
| | PsA | Ps | Controls |
| Characteristic | (n = 16) | (n = 15) | (n = 17) |
| Age, years, mean (median) | 46.2 (40) | 39.4 (37) | 42.2 (39) |
| Female, % | 56% | 53% | 64% |
| Ethnicity, white* | 62% | 66% | 59% |
| HLA-B27, % | 12% | n/a | n/a |
| HLA-Cw6, % | 18% | n/a | n/a |
| HLA-B27 and/or Cw6, % | 30% | n/a | n/a |
| Disease duration, months, | 0.8 (0) | 16 (11) | n/a |
| mean (median) | | | |
| Disease activity parameters | | | |
| (articular) | | | |
| CRP, mg/l, mean (median) | 7.5 (0) | 1 (0) | 0 |
| DAS28, mean (median) | 4.8 (4.7) | n/a | n/a |
| Patient VAS pain, mm, mean | 50.6 (45) | n/a | n/a |
| (median) | | | |
| Active Joint Count, mean | 4.7 (3) | n/a | n/a |
| (median) | | | |
| Axial involvement, % | 25% | n/a | n/a |
| Disease activity parameters | | | |
| (skin) | | | |
| PASI, mean (median) | 5.2 (3.8) | 6.3 (4.3) | n/a |
| Nail Psoriasis, % | 75% | 69% | n/a |
| Medication use | | | |
| NSAIDs, current % | 75% | 0% | n/a |
| Methotrexate, % | 6%** | 0% | n/a |
| Prednisone, % | 0% | 0% | n/a |
| Biological agent, % | 0% | 0% | n/a |

Abbreviations:
PsA, psoriatic arthritis;
Ps, psoriasis of the skin only;
CRP, C-reactive protein;
DAS28, Disease Activity Score with 28 joint count;
VAS, visual analog scale;
PASI, Psoriasis Area and Severity Index;
NSAIDs, non-steroidal anti-inflammatory drugs.
*Including Hispanic whites.
**One patient had received one dose of methotrexate the week prior to enrollment.

Decreased Diversity in PsA and Ps Gut Microbiota.

A total of 48 fecal samples were obtained from PsA, Ps and healthy subjects for sequencing. Using a distance-based similarity of ≧97% for operational taxonomic units (OTU) assignment, a total of 2835 OTUs were identified. When compared to healthy subjects, microbial diversity was significantly reduced in PsA and Ps samples, as calculated by the Shannon diversity index and Faith's phylodiversity index (Supplementary FIGS. 1A, B). Subsequently, the present inventors analyzed whether the overall structure of the microbiota of healthy samples differed from that of Ps and PsA and quantified the similarity by applying the UniFrac phylogenetic distance. PCoA was further applied to cluster samples along orthogonal axes of maximal variance. As shown in Supplementary FIG. 1C, PC1 axes discriminated most healthy samples from the majority of Ps and PsA samples. Moreover, analysis of molecular variance (AMOVA) of the obtained UniFrac distances between samples revealed that overall microbiota structure was also significantly different when comparing PsA to Ps samples (Supplementary FIG. 1).

Lower Relative Abundance of Akkermansia and Ruminoccocus is Characteristic of PsA Gut Microbiota.

To investigate further which bacterial taxa were distinct among groups, LefSe analysis was applied (see Methods). Interestingly, while no bacterial taxa were found to be enriched in PsA patients, relative abundance of several microbial clades were decreased in both PsA and Ps, and therefore enriched in healthy controls (FIG. 1 and Supplementary FIG. 2). Within these identified components of the intestinal microbiota, Akkermansia, Ruminococcus and Pseudobutyrivibrio were considered the most relevant genera that discriminated PsA microbiota from healthy controls (FIG. 1A, D). At other levels of taxonomic classification, unclassified Clostridia and the parental taxonomic levels of Akkermansia (Verrucomicrobia, Verrucomicrobiae and Verrucomicrobiales) were also significantly decreased in PsA. The Ps gut microbiota was characterized by a reduced relative abundance of the genera Parabacteroides and Coprobacillus (FIG. 1B, D). The comparison between PsA and Ps groups revealed that the higher taxonomic levels for Akkermansia and Ruminoccocus (including Firmicutes/Clostridiales and Verrucomicrobiales, respectively) were significantly less abundant in PsA patients (FIG. 1C, D), while Bacteroidetes phylum and Coprobacillus genus were less abundant in Ps samples. Akkermansia and Ruminoccocus per se were also relatively decreased in PsA (FIG. 1C, Supplementary FIG. 2).

The present inventors' ability to analyze the microbiota beyond the genus level facilitated investigation of the various OTUs that were underrepresented in patients with PsA. Several OTUs had a decreased relative abundance compared to healthy controls, including OTUs 43 (Coprococcus), 31 (Pseudobutyrivibrio), 26 (Parabacteroides), 83 (unclassified_{—}Ruminococcaceae), 65 (Alistipes), and 85 (Akkermansia) (FIG. 2A, 2D and Supplementary FIG. 3). Intriguingly, several of these OTUs, including OTU43 as well as OTUs 26, 83, and 35 were also decreased in Ps patients, suggesting a possible common gut microbiota signature for Ps and PsA (FIG. 2 and Supplementary FIG. 3). Moreover, Ps samples showed a significantly decreased relative abundance of several other OTUs when compared to healthy subjects, including OTUs 44, 89 and 106 (FIG. 2B, 2D). OTU11 was the only overrepresented OTU in the Ps group. When comparing PsA and Ps groups, patients with skin disease only had increased relative abundance of OTUs 11, 16 and 32, while patients with PsA had an overrepresentation of OTU44 only (FIG. 2C, Supplementary FIG. 3).

Local Gut Immune Response in PsA is Characterized by an Increase in Fecal sIgA and a Decrease in RANKL Levels.

To investigate further whether this alteration in intestinal microbial communities in PsA or Ps patients was associated with a differential local immune response, luminal concentrations of sIgA were measured in all groups. Given their proposed role in PsA pathogenesis (18), fecal levels of RANKL and OPG were also measured, along with 5100 (a novel neutrophil-derived mucosal marker for IBD). PsA patients had an increase in sIgA relative to healthy controls (FIG. 3A; P=0.06). Conversely, fecal RANKL was significantly reduced in PsA (FIG. 3B; P<0.05), with only 19% of patients having measurable RANKL compared to 30% of healthy subjects and 75% of Ps patients. Fecal OPG was significantly lower in Ps vs controls (FIG. 3C; P<0.05) but not when compared to PsA. Levels of fecal S100 were similar among all groups. To determine whether fecal quantities of all these proteins were a reflection of systemic levels (which may have translocated into the gut lumen), concomitant serum measurements were performed to calculate correlation coefficients. Although there was no significant correlation between fecal and serum levels of these proteins (Supplementary FIG. 4), serum levels of S100 were significantly elevated in Ps patients compared to PsA and healthy subjects (FIG. 3D; P<0.05).

Characteristic Interrelations of Gut Microbiota and Metadata in PsA.

Because of univariate associations between groups, gut microbiota and metadata, the present inventors set out to describe correlations between decreased taxa in Ps and PsA and the various measured fecal and serum proteins. An optimal Bayesian network, which incorporates correlations between taxa, was also performed. The analyses revealed that Akkermansia (as well as OTU85) was inversely correlated with fecal levels of sIgA. Interestingly, Coprobacillus, a genus decreased in Ps, negatively correlated with S100 levels in serum. OTU43 (Coprococcus) correlated with OTU31, which in turn positively correlated with OTU109, all of which were also decreased in both groups of patients. OTU16 (UC_{—}Lachnospiraceae), low in PsA, was the only OTU positively correlated with fecal levels of RANKL (also decreased in PsA), and co-occurred with two other OTUs that were relatively decreased in PsA: OTU32 and OTU119. Taken together, these interactions describe a potential distinctive pattern representative of the PsA gut microbiota, characterized by lower relative abundance of several taxa and decreased levels of fecal RANKL.

Finally, the Bayesian network analysis showed that OTUs 10 (Parabacteroides), 44 and 35 (both UC Lachnospiraceae) also clustered together (Supplementary FIG. 5), with the latter taxa revealing the highest inverse correlation with serum levels of S100 (FIG. 5B). These interactions differentiate the gut microbiota of the Ps cohort and its associations with systemic inflammatory markers.

### DISCUSSION

An expanding body of literature has linked the intestinal microbiota, gut inflammation (both clinical and subclinical), and the different phenotypic expressions of spondyloarthritis.

Two decades ago, seminal work determined the role of gut microbiota in the development of arthritis and colitis in HLA-B27 transgenic rats (7). Since then, many studies have contributed to strengthen this hypothesis, namely that in genetically susceptible subjects a state of gut microbial dysbiosis (alteration in the homeostasis of bacterial composition) promotes an exaggerated immune response in the host's intestinal lamina propria, activating systemic inflammation and ultimately leading to joint disease. The role of HLA-B27 and related genes was recently validated in AS, IBD and PsA (19). Subclinical histological and molecular markers of gut inflammation are also found in patients with AS (20,21) and with PsA (10). While these studies addressed the activation of host mucosal immunity, they did not directly examine the role of the intestinal microbiome. The link between intestinal bacteria and SpA has been investigated separately. Prior studies, however, utilized indirect serologic methods, classic culture approaches and/or limited, low-throughput PCR/DGGE-based techniques (22), assessing for prevalence of only a handful of taxa.

Utilizing high-throughput, culture-independent, 16S rRNA gene pyrosequencing technology the present inventors have shown, for the first time, that patients with PsA and Ps have a decreased diversity in their gut microbiota, mainly due to lower relative abundance of several taxa. Some of this taxa reduction is shared between both conditions [i.e. OTU 43 (Coprococcus)], suggesting a distinctive intestinal gut microbiota that is common in psoriasis of the skin but independent of arthritis. Interestingly, however, other genera such as Ruminoccocus and Akkermansia are uniquely decreased in PsA.

This is intriguing for several reasons. First, similar results have been consistently reported (and replicated) in microbiome studies of patients with IBD, and particularly in those with Crohn's disease (23). Second—and perhaps more remarkable—is the present finding that this relatively lower diversity in PsA and IBD microbiota is mainly driven by a decrease in phylogenetically similar microbiome members. Several reports showed that Akkermansia and Ruminococcus species, as well as Alistipes genus, are also diminished in IBD patients. Akkermansia is detectable in the majority of healthy subjects (24) and is an important component in two of three recently described human gut enterotypes (25). Using publicly available genetic alignment tools (BLAST), the present inventors found that OTU85 has 100% sequence identity to Akkermansia muciniphila and is virtually absent from our PsA cohort. Functionally, A. muciniphila is predominantly a mucus-degrading gut symbiont that converts mucin into short-chain fatty acids (SCFAs) Acetate and Propionate, activating host epithelial cells and stimulating an adequate immune response. Notably, A. muciniphila was the most abundantly identified mucolytic mucosa-associated bacterium in healthy controls vs IBD patients (26-28), suggesting a protective role for this taxon. In two recent studies, Ruminococcaceae were also underrepresented in gut microbiota from IBD patients, particularly in ileal Crohn's disease (29,30). Many Ruminococcus species, except R. gnavus, are decreased in Crohn's disease (31). Interestingly, several Ruminococcus species are also mucin-degrading bacteria and important in maintaining gut homeostasis, particularly via the production of SCFAs. SCFAs in turn promote intestinal health, creating favorable conditions for resistance to pathogenic bacteria and protection against colitis (32). None of the SCFAs analyzed in our cohort, however, were significantly lower in PsA patients.

Other gut bacterial genera were relatively decreased in our PsA cohort, including OTU43 (Coprococcus).

It is plausible, therefore, that a simultaneous reduction in all these taxa translates into functional consequences on the capacity of predisposed subjects to regulate intestinal immune responses. An inability to contain this altered antigenic load may lead to broader inflammation, either in the gut (as in the case of Crohn's disease) or in distal compartments, such as entheses or joints (as in PsA and other types of SpA) (3,5). In the studies described herein, increased immune activation in the intestine that accompanied the dysbiosis was reflected by a higher concentration of sIgA in the gut lumen of PsA patients. It is conceivable that this indicates a local mucosal reactive pattern marked by an effort of the host to confine the immune response to the gut. Conversely, this could also represent the initial evidence of a gut barrier breach only seen in individuals with a specific intestinal dysbiosis, as in the case of recent-onset PsA (i.e., decreased diversity; absence of protective taxa). The exact nature of this response, its antigenic target and its cause-effect directionality merit further study.

Intriguingly, the majority of patients with PsA had selectively low levels of gut lumen RANKL compared to Ps subjects and controls. This observation is relevant for several reasons. First, levels of RANKL have been found to be higher in sera of PsA patients (38,39), while RANKL expression was also up-regulated in PsA synovium (40) and in the epidermis of psoriasis patients (41). Second, gut stromal-derived RANKL is the critical factor controlling the differentiation of microfold (M) cells in the intestinal lamina propria, which are crucial for sampling of antigens from the lumen (42). Lastly, in colonic explant cultures from patients with IBD there was an increase in OPG secretion with normal levels of RANKL (43). It is conceivable that this decrease in fecal RANKL levels in PsA patients may actually represent a response to a specific composition of gut bacterial community. Alternatively, the RANKL/OPG system may be a true modulator of gut microbiota and an imbalance in detriment of RANKL in the lumen of PsA patients may facilitate the dissemination of inflammation to distal (joint) sites.

The present inventors have previously utilized this approach to examine the intestinal microbiome in treatment-naive, new-onset RA (NORA) patients (12), and found that expansion of Prevotella copri, was associated with enhanced susceptibility to yet-untreated human RA. This is in contrast with findings in PsA patients as presented herein and suggests a distinctive pattern for both conditions. The significance of this divergence is unclear. Prevotella may represent an environmental trigger for RA in at-risk individuals (e.g., those with circulating autoantibodies) as it has been found to exacerbate induced colitis in mice by our group (12) and others (44). A second possibility is that Prevotella expands preferentially under specific local and systemic inflammatory conditions, representing rather a downstream consequence of RA pathogenesis. These marked differences will require further validation and mechanistic investigations as they may significantly contribute to the understanding of RA and Ps-PsA pathogenesis.

A key question left unanswered is whether patients with current psoriasis of the skin alone (Ps) will lose certain potentially protective taxa-such as Akkermansia and Ruminoccocus—at the time of (or prior to) transition into PsA. This is crucial because, although it is established that 25-30% of Ps patients will develop arthritis over time, there is currently no way to predict progression. A prospective “natural history” cohort of individuals with Ps is ongoing in our center and should help to answer some of these questions.

In summary, the present studies constitute a novel and comprehensive approach to investigate the symbiotic relationship between gut microbiota and PsA. The present inventors have identified several organisms that are practically absent from PsA patients (i.e., Akkermansia, Ruminococcus), resembling a state of gut dysbiosis previously described in IBD subjects. These taxa and the associated changes in immune response merit further study as potential modulators of autoimmunity in both conditions. The Ps gut microbiota profile appears to be intermediate between PsA and healthy subjects, suggesting a possible continuum in disappearing intestinal taxa through the natural history of disease.

### REFERENCES

1. Eder L, Chandran V, Pellet F, Shanmugarajah S, Rosen C F, Bull S B, et al. Human leucocyte antigen risk alleles for psoriatic arthritis among patients with psoriasis. Ann Rheum Dis 2012; 71:50-5.
2. Winchester R, Minevich G, Steshenko V, Kirby B, Kane D, Greenberg D A, et al. HLA associations reveal genetic heterogeneity in psoriatic arthritis and in the psoriasis phenotype. Arthritis Rheum 2012; 64:1134-44.
3. Cua D J, Sherlock J P. Autoimmunity's collateral damage: Gut microbiota strikes ‘back’. Nat Med 2011; 17:1055-6.
4. Van Praet L, Van den Bosch F, Mielants H, Elewaut D. Mucosal inflammation in spondylarthritides: past, present, and future. Curr Rheumatol Rep 2011; 13:409-15.
5. Sherlock J P, Joyce-Shaikh B, Turner S P, Chao C C, Sathe M, Grein J, et al. IL-23 induces spondyloarthropathy by acting on ROR-gammat+CD3+CD4-CD8-entheseal resident T cells. Nat Med 2012; 18:1069-76.
6. Van Praet L, Van den Bosch F E, Jacques P, Carron P, Jans L, Colman R, et al. Microscopic gut inflammation in axial spondyloarthritis: a multiparametric predictive model. Ann Rheum Dis 2013; 72:414-7.
7. Rath H C, Herfarth H H, Ikeda J S, Grenther W B, Hamm T E, Jr., Balish E, et al. Normal luminal bacteria, especially Bacteroides species, mediate chronic colitis, gastritis, and arthritis in HLA-B27/human beta2 microglobulin transgenic rats. J Clin Invest 1996; 98:945-53.
8. Ruutu M, Thomas G, Steck R, gli-Esposti M A, Zinkernagel M S, Alexander K, et al. beta-glucan triggers spondylarthritis and Crohn's disease-like ileitis in SKG mice. Arthritis Rheum 2012; 64:2211-22.
9. Orchard T R, Wordsworth B P, Jewell D P. Peripheral arthropathies in inflammatory bowel disease: their articular distribution and natural history. Gut 1998; 42:387-91.
10. Scarpa R, Manguso F, D'Arienzo A, D'Armiento F P, Astarita C, Mazzacca G, et al. Microscopic inflammatory changes in colon of patients with both active psoriasis and psoriatic arthritis without bowel symptoms. J Rheumatol 2000; 27:1241-6.
11. Li W Q, Han J L, Chan A T, Qureshi A A. Psoriasis, psoriatic arthritis and increased risk of incident Crohn's disease in US women. Ann Rheum Dis 2013; 72:1200-5.
12. Scher J U, Sczesnak A, Longman R S, Segata N, Ubeda C, Bielski C, et al. Expansion of intestinal Prevotella copri correlates with enhanced susceptibility to arthritis. Elife 2013; 2:e01202.
13. Scher J U, Ubeda C, Equinda M, Khanin R, Buischi Y, Viale A, et al. Periodontal disease and the oral microbiota in new-onset rheumatoid arthritis. Arthritis Rheum 2012; 64:3083-94.
14. Sheneman L, Evans J, Foster J A. Clearcut: a fast implementation of relaxed neighbor joining. Bioinformatics 2006; 22:2823-4.
15. Nielsen O H, Gionchetti P, Ainsworth M, Vainer B, Campieri M, Borregaard N, et al. Rectal dialysate and fecal concentrations of neutrophil gelatinase-associated lipocalin, interleukin-8, and tumor necrosis factor-alpha in ulcerative colitis. Am J Gastroenterol 1999; 94:2923-8.
16. Segata N, Izard J, Waldron L, Gevers D, Miropolsky L, Garrett W S, et al. Metagenomic biomarker discovery and explanation. Genome Biol 2011; 12:R60.
17. Scurati M. Learning Bayesian Networks with the bnlearn R Package. Journal of Statistical Software 2010; 35(3):1-22.
18. Mensah K A, Ritchlin C T, Schwarz E M. RANKL induces heterogeneous DC-STAMP(lo) and DC-STAMP(hi) osteoclast precursors of which the DC-STAMP(lo) precursors are the master fusogens. J Cell Physiol 2010; 223:76-83.
19. Reveille J D. Genetics of spondyloarthritis—beyond the MHC. Nat Rev Rheumatol 2012; 8:296-304.
20. Mielants H, Veys E M, Cuvelier C, De Vos M, Goemaere S, De Clercq L, et al. The evolution of spondyloarthropathies in relation to gut histology. II. Histological aspects. J Rheumatol 1995; 22:2273-8.
21. Ciccia F, Accardo-Palumbo A, Alessandro R, Rizzo A, Principe S, Peralta S, et al. Interleukin-22 and interleukin-22-producing NKp44+ natural killer cells in subclinical gut inflammation in ankylosing spondylitis. Arthritis Rheum 2012; 64:1869-78.
22. Stebbings S, Munro K, Simon M A, Tannock G, Highton J, Harmsen H, et al. Comparison of the faecal microflora of patients with ankylosing spondylitis and controls using molecular methods of analysis. Rheumatology (Oxford) 2002; 41:1395-401.
23. Manichanh C, Rigottier-Gois L, Bonnaud E, Gloux K, Pelletier E, Frangeul L, et al. Reduced diversity of faecal microbiota in Crohn's disease revealed by a metagenomic approach. Gut 2006; 55:205-11.
24. Collado M C, Derrien M, Isolauri E, de Vos W M, Salminen S. Intestinal integrity and Akkermansia muciniphila, a mucin-degrading member of the intestinal microbiota present in infants, adults, and the elderly. Appl Environ Microbiol 2007; 73:7767-70.
25. Arumugam M, Raes J, Pelletier E, Le Paslier D, Yamada T, Mende D R, et al. Enterotypes of the human gut microbiome. Nature 2011; 473(7346):174-80.
26. Png C W, Linden S K, Gilshenan K S, Zoetendal E G, McSweeney C S, Sly L I, et al. Mucolytic bacteria with increased prevalence in IBD mucosa augment in vitro utilization of mucin by other bacteria. Am J Gastroenterol 2010; 105:2420-8.
27. Vigsnaes L K, Brynskov J, Steenholdt C, Wilcks A, Licht T R. Gram-negative bacteria account for main differences between faecal microbiota from patients with ulcerative colitis and healthy controls. Benef Microbes 2012; 3:287-97.
28. Rajilic-Stojanovic M, Shanahan F, Guarner F, de Vos W M. Phylogenetic analysis of dysbiosis in ulcerative colitis during remission. Inflamm Bowel Dis 2013; 19:481-8.
29. Willing B P, Dicksved J, Halfvarson J, Andersson A F, Lucio M, Zheng Z, et al. A pyrosequencing study in twins shows that gastrointestinal microbial profiles vary with inflammatory bowel disease phenotypes. Gastroenterology 2010; 139:1844-54.
30. Morgan X C, Tickle T L, Sokol H, Gevers D, Devaney K L, Ward D V, et al. Dysfunction of the intestinal microbiome in inflammatory bowel disease and treatment. Genome Biol 2012; 13:R79.
31. Joossens M, Huys G, Cnockaert M, De Preter V, Verbeke K, Rutgeerts P, et al. Dysbiosis of the faecal microbiota in patients with Crohn's disease and their unaffected relatives. Gut 2011; 60:631-7.
32. Smith P M, Howitt M R, Panikov N, Michaud M, Gallini C A, Bohlooly Y, et al. The microbial metabolites, short-chain fatty acids, regulate colonic Treg cell homeostasis. Science 2013; 341(6145):569-73.
33. Garner C E, Smith S, de Lacy C B, White P, Spencer R, Probert C S, et al. Volatile organic compounds from feces and their potential for diagnosis of gastrointestinal disease. FASEB J 2007; 21:1675-88.
34. De Preter V, Machiels K, Joossens M, Arijs I, Matthys C, Vermeire S, et al. Faecal metabolite profiling identifies medium-chain fatty acids as discriminating compounds in IBD. Gut 2014. [Epub ahead of print]
35. Liberato M V, Nascimento A S, Ayers S D, Lin J Z, Cvoro A, Silveira R L, et al. Medium chain fatty acids are selective peroxisome proliferator activated receptor (PPAR) gamma activators and pan-PPAR partial agonists. PLoS One 2012; 7(5):e36297.
36. Bassaganya-Riera J, Viladomiu M, Pedragosa M, De S C, Carbo A, Shaykhutdinov R, et al. Probiotic bacteria produce conjugated linoleic acid locally in the gut that targets macrophage PPAR gamma to suppress colitis. PLoS One 2012; 7(2):e31238.
37. Bassaganya-Riera J, Hontecillas R, Home W T, Sandridge M, Herfarth H H, Bloomfeld R, et al. Conjugated linoleic acid modulates immune responses in patients with mild to moderately active Crohn's disease. Clin Nutr 2012; 31(5):721-7.
38. Chandran V, Cook R J, Edwin J, Shen H, Pellett F J, Shanmugarajah S, et al. Soluble biomarkers differentiate patients with psoriatic arthritis from those with psoriasis without arthritis. Rheumatology (Oxford) 2010; 49:1399-405.
39. Xue Y, Jiang L, Cheng Q, Chen H, Yu Y, Lin Y, et al. Adipokines in psoriatic arthritis patients: the correlations with osteoclast precursors and bone erosions. PLoS One 2012; 7(10):e46740.
40. Ritchlin C T, Haas-Smith S A, Li P, Hicks D G, Schwarz E M. Mechanisms of TNF-alpha- and RANKL-mediated osteoclastogenesis and bone resorption in psoriatic arthritis. J Clin Invest 2003; 111:821-31.
41. Toberer F, Sykora J, Gottel D, Ruland V, Hartschuh W, Enk A, et al. Tissue microarray analysis of RANKL in cutaneous lupus erythematosus and psoriasis. Exp Dermatol 2011; 20:600-2.
42. Knoop K A, Kumar N, Butler B R, Sakthivel S K, Taylor R T, Nochi T, et al. RANKL is necessary and sufficient to initiate development of antigen-sampling M cells in the intestinal epithelium. J Immunol 2009; 183:5738-47.
43. Moschen A R, Kaser A, Enrich B, Ludwiczek O, Gabriel M, Obrist P, et al. The RANKL/OPG system is activated in inflammatory bowel disease and relates to the state of bone loss. Gut 2005; 54:479-87.
44. Elinav E, Strowig T, Kau A L, Henao-Mejia J, Thaiss C A, Booth C J, et al. NLRP6 inflammasome regulates colonic microbial ecology and risk for colitis. Cell 2011; 145:745-57.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

1. A method for treating a subject afflicted with psoriasis, the method comprising administering to the subject a therapeutically effective amount of a sample comprising a fecal transplant isolated from a healthy donor or a therapeutically effective amount of a composition comprising at least one purified bacterial species of a genera of Akkermansia, Ruminococcus, Pseudobutyrivibrio, Coprococcus, and Coprobacillus, wherein administering the fecal transplant or the composition comprising the at least one purified bacterial species treats the subject afflicted with psoriasis.

2. The method of claim 1, wherein the subject afflicted with psoriasis has decreased bacterial diversity of gut microbiota.

3. The method of claim 1, wherein the subject afflicted with psoriasis exhibits patches of thick, inflamed skin covered with silvery scales; and does not exhibit abdominal pain, vomiting, diarrhea, rectal bleeding, severe pelvic cramps and/or weight loss; and does not exhibit pain, swelling, or stiffness in one or more joints, joints that are red or warm to the touch, dactylitis, pain in and around the feet and ankles, and/or lower back pain.

4-6. (canceled)

7. The method of claim 1, wherein the sample comprising the fecal transplant is administered orally or anally.

8. (canceled)

9. The method of claim 1, wherein the at least one purified bacterial species is selected from the group consisting of Akkermansia muciniphila (A. muciniphila), Ruminococcus albus (R. albus), Ruminococcus callidus (R. callidus), Ruminococcus bromii (R. bromii), and Ruminococcus gnavus (R. gnavus).

10. The method of claim 1, wherein the Coprococcus species is ATCC®27761™; ATCC®27758™; or ATCC®27759™; the Ruminococcus species is Ruminococcus gnavus (ATCC®BAA-35913™) or Ruminococcus gnavus (ATCC®BAA-29149™); and/or the Akkermansia species is Akkermansia muciniphila (ATCC®BAA-835™).

11. The method of claim 1, wherein the composition comprises a combination of purified A. muciniphila and purified R. gnavus; a combination of purified A. muciniphila, purified R. gnavus, and a purified species of Coprococcus; or a combination of purified A. muciniphila, purified R. gnavus, and a purified species of Pseudobutyrivibrio; a combination of purified A. muciniphila, purified R. gnavus, a purified species of Coprococcus, and a purified species of Pseudobutyrivibrio.

12-16. (canceled)

17. The method of claim 2, wherein the decreased bacterial diversity of gut microbiota is determined by analyzing the gut microbiota of the subject by nucleic acid sequencing.

18. The method of claim 17, wherein the nucleic acid sequencing is shotgun sequencing or 16s rRNA sequencing.

19. A composition for treating PsA in a subject, the composition comprising a therapeutically effective amount of at least one of a purified bacterial species of Akkermansia, Ruminococcus, Pseudobutyrivibrio, Coprococcus, Coprobacillus, Unclassified (UC)_{—}Clostridia, Verrucomicrobiae, Verrucomicrobia, and Verrucomicrobiales and a sterile physiologically compatible carrier or excipient.

20-28. (canceled)

29. A method for treating a subject afflicted with psoriatic arthritis, the method comprising administering to the subject a therapeutically effective amount of a sample comprising a fecal transplant isolated from a healthy donor or a therapeutically effective amount of a composition comprising at least one purified bacterial species of Akkermansia, Ruminococcus, Pseudobutyrivibrio, Coprococcus, Coprobacillus, Unclassified (UC)_{—}Clostridia, Verrucomicrobiae, Verrucomicrobia, and Verrucomicrobiales, wherein administering the fecal transplant or the composition treats the subject afflicted with psoriatic arthritis.

30. A method for treating a subject afflicted with psoriasis, the method comprising administering to the subject a therapeutically effective amount of a sample comprising a fecal transplant isolated from a healthy donor or a therapeutically effective amount of a composition comprising at least one of a purified bacterial species of Coprococcus, Coprobacillus, Porphyromonadaceae, Parabacteroides, Unclassified (UC)_{—}Clostridia, Erysipelotrichi, and Actinobacteria phylum, wherein administering the fecal transplant or the composition treats the subject afflicted with psoriasis.

31. The method of claim 30, wherein the subject afflicted with psoriasis has decreased bacterial diversity of gut microbiota.

32. The method of claim 29, wherein the subject afflicted with psoriatic arthritis exhibits psoriasis of the skin associated with: pain, swelling, or stiffness in one or more joints, joints that are red or warm to the touch, dactylitis, pain in and around the feet and ankles, and/or lower back pain and does not exhibit abdominal pain, vomiting, diarrhea, rectal bleeding, severe pelvic cramps and/or weight loss.

33. The method of claim 30, wherein the subject afflicted with psoriasis exhibits patches of thick, inflamed skin covered with silvery scales; and does not exhibit abdominal pain, vomiting, diarrhea, rectal bleeding, severe pelvic cramps and/or weight loss; and does not exhibit pain, swelling, or stiffness in one or more joints, joints that are red or warm to the touch, dactylitis, pain in and around the feet and ankles, and/or lower back pain.

34. The method of claim 30, wherein the healthy donor is an individual without any chronic medical conditions.

35. The method of claim 30, wherein the sample comprising the fecal transplant is administered orally or anally.

36. (canceled)

37. The method of claim 30, wherein the Coprococcus species is ATCC®27761™; ATCC®27758™; or ATCC®27759™.

38-48. (canceled)


SEQUENCE LISTING (2 sequences)

SEQ ID NO: 1 - DNA, 47 bp, Artificial Sequence
  Synthetic oligonucleotide
ctatgcgccttgccagcccgctcagtcagagtttgatcctggctcag

SEQ ID NO: 2 - DNA, 57 bp, Artificial Sequence
  Synthetic oligonucleotide
cgtatcgcctccctcgcgccatcagnnnnnnnnnnnncagctgcctcccgtaggagt